(19) United States
(12) Patent Application Publication
(10) Pub. No.: US 2019/0051409 A1
(43) Pub. Date: Feb. 14, 2019

(54) MULTI-DEVICE SYSTEM

(71) Applicant: Neural Analytics, Inc., Los Angeles, CA (US)

(72) Inventors: Leo Petrossian, Los Angeles, CA (US); Jan Zwierstra, Los Angeles, CA (US); Richard Castro, Los Angeles, CA (US); Lane Stith, Los Angeles, CA (US)

(21) Appl. No.: 16/101,332

(22) Filed: Aug. 10, 2018

Related U.S. Application Data
(60) Provisional application No. 62/544,362, filed on Aug. 11, 2017.

(51) Int. Cl. G16H 40/60; A61B 5/00; G06F 3/01

(52) U.S. Cl. CPC G16H 40/60; A61B 5/0024; A61B 5/0036; A61B 5/0035; G06F 3/011

(57) ABSTRACT

Arrangements described herein relate to a multi-device system configured to provide healthcare to a subject. The multi-device system includes a multi-device having a body interface, a plurality of devices fixed to the body interface, each of the plurality of devices configured to collect data of the subject, and a body operatively coupled to the multi-device at the body interface. The body is configured to position the multi-device with respect to the subject. The body interface is configured to move relative to the body to selectively activate at least one of the plurality of devices.

### CROSS-REFERENCE TO RELATED PATENT APPLICATION

This application claims the benefit of and priority to U.S. provisional patent application Ser. No. 62/544,362, filed Aug. 11, 2017, the contents of which are incorporated herein by reference in its entirety.

### BACKGROUND

To provide more efficient and effective healthcare, a variety of different medical devices can be utilized to diagnose and treat a subject (e.g., a patient). For example, if the subject is experiencing health problems that are not readily diagnosable, a variety of imaging and diagnosing devices can assist a healthcare provider in determining the undiagnosed condition. However, use of multiple different medical devices can be cumbersome and even impractical for a healthcare provider, especially in an emergency situation where time of diagnosing and administering treatment to a subject is critical for the subject's health. In addition, these medical devices often are operated by highly skilled technicians, thus further complicating the administration of healthcare.

### SUMMARY

According to various arrangements, a multi-device system can provide efficient and effective healthcare administration such that a highly skilled technician is not needed to operate the multi-device system. For example, the multi-device system can readily provide multiple different imaging and physiological scanning devices for implementation with respect to a subject (e.g., a patient, an animal, and the like), as the multi-device system can easily toggle between the different devices. Therefore, more efficient and effective administration of healthcare can be provided by the automation achieved by the multi-device system.

In some arrangements, a multi-device system for providing healthcare to a subject includes a multi-device, which includes a body interface and a plurality of devices fixed to the body interface. Each of the plurality of devices configured to collect data of or provide therapy to the subject. The multi-device system further includes a body operatively coupled to the multi-device at the body interface. The body is configured to position the multi-device with respect to the subject. The body interface is configured to move relative to the body to selectively activate at least one of the plurality of devices.

In some arrangements, each of the plurality of devices is configured to collect data of the subject and includes at least one of a camera, an ultrasound range device, an ultrasound imaging device, a thermal imaging device, a near-infrared imaging device, a near-infrared spectroscopy device, an optical camera device, a lighting device, a spectrometer device, a microphone device, an electroencephalography device, and a metal detector.

In some arrangements, each of the plurality of devices is configured to provide therapy to the subject and includes at least one of a laser, a syringe, an ablation device, a light therapy device, an electric shock therapy device, a stitching device, a scalpel, a catheter, an insulin pump, a bandaging device, and a respirator.

In some arrangements, the multi-device system of claim 1 further includes a power supply configured to provide power to the plurality of devices and a controller configured to transmit operational instructions to the plurality of devices and receive the data collected by the plurality of devices. The at least one of the plurality of devices is activated when the at least one of the plurality of devices is receiving the power and the operational instructions.

In some arrangements, the body interface includes a first connection interface operatively coupled to each of the plurality of devices. The body includes a second connection interface operatively coupled to the power supply and the controller. The second connection interface is configured to provide the power and the operational instructions to the first connection interface for the activated at least one of the plurality of devices by operatively coupling to the first connection interface when the body interface is moved relative to the body to a predetermined position. The first connection interface is configured to provide the data collected by the at least one of the plurality of devices to the second connection interface by operatively coupling to the second connection interface.

In some arrangements, the first connection interface for the at least one of the plurality of devices includes a first power interface and a first data communication interface separate from the first power interface. The second connection interface includes a second power interface and a second data communication interface separate from the second power interface. The second power interface is configured to operatively couple to the first power interface when the body interface is moved relative to the body to the predetermined position to provide the power to the first power interface. The second data communication interface is configured to operatively couple to the first data communication interface when the body interface is moved relative to the body to the predetermined position to provide the operational instructions to the first data communication interface and to receive the data collected by the activated at least one of the plurality of devices from the first data communication interface.

In some arrangements, the second connection interface is configured to provide the power and the operational instructions to the first connection interface by providing to the first connection interface modulated power signals includes the power from the power supply and the operational instructions from the controller.

In some arrangements, the body interface includes a locking device configured to structurally maintain the predetermine position.

In some arrangements, the body interface includes a first connection interface operatively coupled to each of the plurality of devices. The body includes a second connection interface operatively coupled to the power supply and the controller. The second connection interface includes at least one data transmission ring configured to receive the data collected by the at least one of the plurality of devices.

In some arrangements, the body interface being configured to move relative to the body to selectively activate at least one of the plurality of devices by: responsive to the body interface being moved relative to the body to a first predetermined position, a first device of the plurality of devices is activated while a second device of the plurality of devices is inactivated; and responsive to the body interface being moved relative to the body to a second predetermined position, the second device is activated while the first device is inactivated.

In some arrangements, the body interface is configured to rotate relative to the body to selectively activate the at least one of the plurality of devices.

In some arrangements, the at least one of the plurality of devices includes a force controller configured to determine a force exerted by the at least one of the plurality of devices against the subject.

In some arrangements, the multi-device further includes a plurality of tools. The plurality of tools is configured to move relative to the plurality of devices. The first tool of the plurality of tools is configured to operatively couple to one of the at least one of the plurality of devices when the plurality of tools is moved to a first relative position relative to the plurality of devices. The first tool being operatively coupled to the one of the at least one of the plurality of devices enables the one of the at least one of the plurality of devices to output first data. A second tool of the plurality of tools is configured to operatively couple to the one of the at least one of the plurality of devices when the plurality of tools is moved to a second relative position relative to the plurality of devices. The second tool being operatively coupled to the one of the at least one of the plurality of devices enables the one of the at least one of the plurality of devices to output second data different from first data.

In some arrangements, the one of the plurality of devices is one of a photo-sensor, an infrared sensor, a particle detector, and a laser emitter. Each of the plurality of tools is at least one of a lens, a photo-projector, and a waveguide.

In some arrangements, the one of the plurality of devices is a photo-sensor. The first tool is a wide-angle lens. The first data include first image data capturing a first portion of the subject. The second tool is a zoom lens. The second data include second image data capturing a second portion of the subject. The first portion is larger than the second portion.

In some arrangements, another one of the plurality of devices is a laser emitter. The plurality of tools includes a grid lens configured to be operatively coupled to the laser emitter in the first relative position. The laser emitter is configured to project a grid onto the subject while the first data is being collected by the photo-sensor through the wide-angle lens or while the second data is being collected by the photo-sensor through the zoom lens.

In some arrangements, the body includes a robotic manipulator having at least two arm segments and a joint. The robotic manipulator is configured to position the multi-device with respect to the subject.

In some arrangements, the body further includes at least one of a mounting mechanism configured to detachably mount the multi-device system to an object, a battery, and a network device.

In some arrangements, the body further includes a manual interface configured to allow the multi-device to be moved relative to the subject by a user of the multi-device system.

In some arrangements, the body further includes a semi-automatic switch. The semi-automatic switch being in a first state corresponds to allowing the multi-device to be moved relative to the subject by the user via the manual interface. The semi-automatic switch being in a second state corresponds to disallowing the multi-device to be moved relative to the subject by the user.

In some arrangements, a method for providing healthcare to a subject using a multi-device system, the method includes providing a multi-device that includes a body interface and a plurality of devices fixed to the body interface, wherein each of the plurality of devices is configured to provide healthcare to the subject by collecting data. The method further includes providing a body operatively coupled to the multi-device at the body interface. The body is configured to position the multi-device with respect to the subject. The body interface is configured to move relative to the body to selectively activate at least one of the plurality of devices.

In some arrangements, the method further includes providing a plurality of tools. The plurality of tools is configured to move relative to the plurality of devices. A first tool of the plurality of tools is configured to operatively couple to a first device of the at least one of the plurality of devices when the plurality of tools is moved to a first relative position relative to the plurality of devices. The first tool being operatively coupled to the first device enables the first device to output first data. A second tool of the plurality of tools is configured to operatively couple to the first device when the plurality of tools is moved to a second relative position relative to the plurality of devices. The second tool being operatively coupled to the first device enables the first device to output second data different from first data.

In some arrangements, the first device and a second device of the at least one of the plurality of devices are configured to be activated simultaneously.

In some arrangements, a third tool of the plurality of tools is configured to operatively couple to the second device when the plurality of tools is moved to the first relative position relative to the plurality of devices.

In some arrangements, the first device is a photo-sensor. The first tool is a wide-angle lens. The second device is a laser emitter. The second tool is a grid lens.

In some arrangements, a method for providing healthcare to a subject using multi-device system having a multi-device and a body configured to move the multi-device relative to the subject includes determining that a semi-automatic movement feature is enabled, in response to determining that the semi-automatic movement feature is enabled, allowing the multi-device to be moved by a user to a first position relative to the subject, performing first registration with respect to a first portion of a body of the subject in the first position, and performing first subject imaging with respect to the first portion of the body of the subject based on the first registration.

In some arrangements, allowing the multi-device to be moved relative to the subject by the user includes providing a manual interface on the body or the multi-device, wherein the manual interface is configured to interact with the user to allow the user to move the multi-device, and enabling the user to move the multi-device by loosening one or more joints on the body.

In some arrangements, the method further includes allowing the multi-device to be moved by the user to a second position relative to the subject, performing second registration with respect to a second portion of a body of the subject in the second position, and performing second subject imaging with respect to the second portion of the body of the subject based on the second registration.

In some arrangements, the first portion is larger in size than the second portion.

In some arrangements, the first portion and the second portion correspond to different body parts of the subject.

In some arrangements, a method for providing healthcare to a subject using a multi-device system having a controller, a multi-device, and a body configured to move the multi-device relative to the subject, the multi-device having a first device and a second device, the method includes determining imaging data for a portion of the subject by scanning, using the first device, the portion of the subject, determining image projection corresponding to the imaging data by processing the imaging data using the controller, and projecting, using the second device, the image projection unto the subject.

In some arrangements, the image projection is projected using the second device unto the portion of the subject or a different portion of the subject.

In some arrangements, the method further includes performing registration with respect to the portion of the subject using a third device of the multi-device, wherein the imaging data is generated based on the registration.

### BRIEF DESCRIPTION OF THE FIGURES

Features, aspects, and advantages of the present invention will become apparent from the following description and the accompanying example arrangements shown in the drawings, which are briefly described below.

FIG. 1 is a system block diagram illustrating an example of a multi-device system according to various arrangements.

FIG. 2A illustrates a front view of a multi-device according to various arrangements.

FIG. 2B illustrates a perspective view of the multi-device shown in FIG. 2A according to various arrangements.

FIG. 2C illustrates a side view of the multi-device shown in FIG. 2A according to various arrangements.

FIG. 2D illustrates a rear view of the multi-device shown in FIG. 2A according to various arrangements.

FIG. 2E illustrates a side view of the multi-device shown in FIG. 2A according to various arrangements.

FIG. 2F illustrates a perspective view of the multi-device shown in FIG. 2A according to various arrangements.

FIG. 2G illustrates a perspective view of the multi-device shown in FIG. 2A according to various arrangements.

FIG. 2H illustrates a perspective view of the multi-device shown in FIG. 2A according to various arrangements.

FIG. 3A illustrates a perspective view of a multi-device system according to various arrangements.

FIG. 3B illustrates a front view of the multi-device system shown in FIG. 3A according to various arrangements.

FIG. 3C illustrates a side view of the multi-device system shown in FIG. 3A according to various arrangements.

FIG. 3D illustrates a top view of the multi-device system shown in FIG. 3A according to various arrangements.

FIG. 3E illustrates a side view of the multi-device system shown in FIG. 3A according to various arrangements.

FIG. 3F illustrates an overhead view of the multi-device system shown in FIG. 3A according to various arrangements.

FIG. 3G illustrates a rear view of the multi-device system shown in FIG. 3A according to various arrangements.

FIG. 3H illustrates a bottom view of the multi-device system shown in FIG. 3A according to various arrangements.

FIG. 4A illustrates a first connection interface of a body interface of a multi-device according to various arrangements.

FIG. 4B illustrates a second connection interface of a body according to various arrangements.

FIG. 5A illustrates a first connection interface of a body interface of a multi-device according to various arrangements.

FIG. 5B illustrates a second connection interface of a body according to various arrangements.

FIG. 6 illustrates tools being operatively coupled to devices according to various arrangements.

FIG. 7 is a flow diagram illustrating a method for using a multi-device system according to various arrangements.

FIG. 8 is a flow diagram illustrating a method for using a multi-device system according to various arrangements.

FIG. 9 illustrates an example implementation of a multi-device system according to various arrangements.

FIG. 10 illustrates an example implementation of a multi-device system according to various arrangements.

### DETAILED DESCRIPTION

The detailed description set forth below in connection with the appended drawings is intended as a description of various configurations and is not intended to represent the only configurations in which the concepts described herein may be practiced. The detailed description includes specific details for providing a thorough understanding of various concepts. However, it will be apparent to those skilled in the art that these concepts may be practiced without these specific details. In some instances, well-known structures and components are shown in block diagram form in order to avoid obscuring such concepts.

In the following description of various arrangements, reference is made to the accompanying drawings which form a part hereof and in which are shown, by way of illustration, specific arrangements in which the arrangements may be practiced. It is to be understood that other arrangements may be utilized, and structural changes may be made without departing from the scope of the various arrangements disclosed in the present disclosure.

As referred to herein, healthcare and the provision thereof refer to one or more of improving, maintaining, and restoring of a subject's health through monitoring, diagnosis, treatment, or another suitable medical procedure. In some examples, the devices, tools, sensors, instruments, apparatuses, systems, and the like are configured to provide healthcare to a subject (e.g., a human patient, an animal, and the like), as described herein.

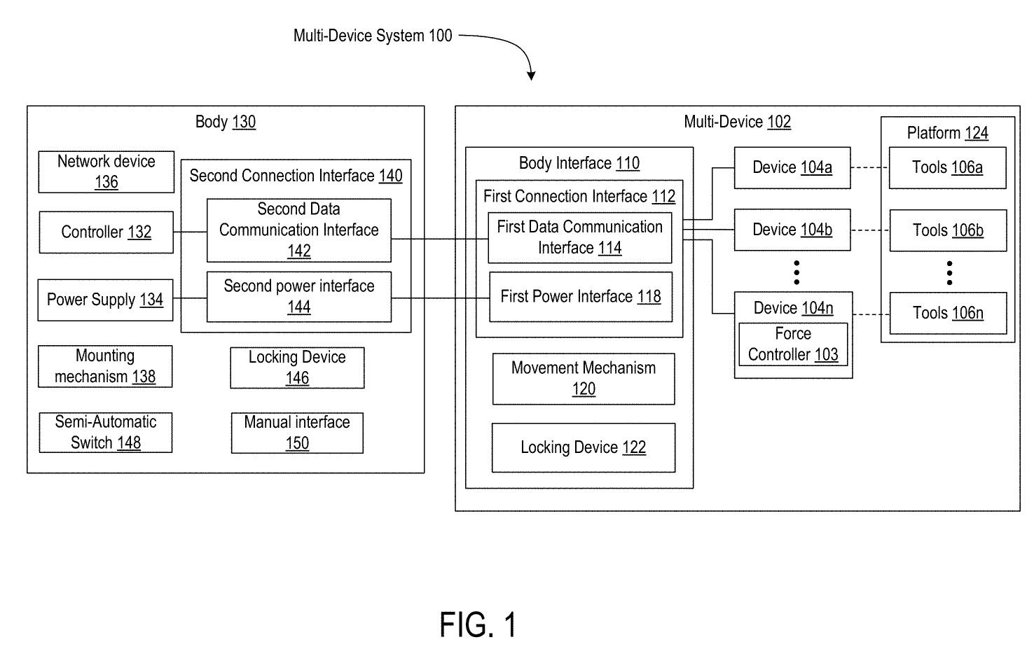

FIG. 1 is a system block diagram illustrating an example of a multi-device system 100 according to various arrangements. Referring to FIG. 1, the multi-device system 100 is configured to provide healthcare to a subject, by autonomously or automatically operating a variety of devices 104a-104n and tools 106a-106n to monitor, diagnose, treat, or perform another suitable medical procedure on a subject. The multi-device system 100 is shown to include at least a multi-device 102 and a body 130.

The multi-device 102 includes multiple devices (e.g., devices 104a-104n). While the multi-device 102 is shown to include the devices 104a-104n, one of ordinary skill in the art can appreciate that the multi-device 102 may have any number of two or more devices disposed thereon. Each of the devices 104a-104n is configured to collect data (e.g., physiological data, biometric data, imaging data, and the like) of a subject. In that regard, each of the devices 104a-104n is or includes at least one of a camera, an ultrasound range device, an ultrasound imaging device, a thermal imaging device, a near-infrared imaging device, a near-infrared spectroscopy device, an optical camera device, a lighting device, a spectrometer device, a microphone device, an electroencephalography device, a metal detector, and any other similar data-gathering device.

The multi-device 102 includes a body interface 110. The devices 104a-104n are fixed, attached, mounted, or otherwise disposed on the body interface 110, such that the devices 104a-104n can move with the body interface 110. In some arrangements, the devices 104a-104n are detachably mounted to the body interface 110, such that one or more of the devices 104a-104n can be detached and replaced with other devices or other types of devices. In some arrangements, the devices 104a-104n are fixed to the body interface 110 such that the devices 104a-104n are immobile relative to the body interface 110. In some arrangements, the body interface 110 further comprises mechanical actuators (not shown) configured to move (e.g., extend, re-orient, or rotate) one or more of the devices 104a-104n in one to six degrees-of-freedom (DOFs) relative to the body interface 110 to enable movement of the one or more of the devices 104a-104n relative to the body interface 110 during data collection. Each mechanical actuator can enable at least one of a linear actuation in the Cartesian system, swivel, sweep, pitch, roll, yaw, and the like. The devices 104a-104n are spaced apart from one another in some arrangements to avoid interference during the operations of the devices 104a-104n.

The body 130 is configured to position the multi-device 102 (e.g., to position the devices 104a-104n and the tools 106a-106n) with respect to a subject. For example, the body 130 includes a robotic manipulator or arm configured to position the multi-device 102 with respect to the subject. In that regard, the robotic manipulator includes arm segments (e.g., at least two arm segments) and joints, each of which connect two arm segments. Each joint is configured to enable one arm segment to move in up to six DOFs (e.g., in at least one of linear actuation in the Cartesian system, swivel, sweep, pitch, roll, yaw, and the like) relative to another arm segment or relative to a platform/base. In that regard, the arm segments and the joints define a workspace or a work envelope for the robotic manipulator. The robotic manipulator can be implemented in any suitable manner to correspond to the operations of the devices 104a-104n and the tools 106a-106n.

The body 130 is configured to be operatively coupled to the multi-device 102 at the body interface 110. The multi-device 102 can be movably supported by the body 130 via the body interface 110. For example, the body interface 110 includes a movement mechanism 120 configured to move the body interface 110 and the entire multi-device 102 relative to the body 130. Particularly, the movement mechanism 120 is configured to move the body interface 110 and the entire multi-device 102 relative to an end of the body 130 that is operatively coupled to the body interface 110. In that regard, the movement mechanism 120 is a mechanical joint that enables the body interface 110 to move in up to six DOFs (e.g., in at least one of linear actuation in the Cartesian system, swivel, sweep, pitch, roll, yaw, and the like) relative to the end of the body 130 that is operatively coupled to the body interface 110. For example, the movement mechanism 120 may include a motor assembly to move the body interface 110 relative to the body 130. Alternatively, instead of disposing the movement mechanism 120 on the body interface 110, a movement mechanism similar to that described herein can be disposed on the body 130 to move the end of the body 130 operatively coupled to the body interface 110 relative to the body interface 110.

In some arrangements, the body interface 110 (and the entire multi-device 102) is detachably mounted to the body 130, such that the multi-device 102 can be detached and replaced with another multi-device.

The multi-device system 100 (e.g., the body 130) includes a power supply 134 configured to provide power to the devices 104a-104n, the tools 106a-106n, the movement mechanism 120, and other components of the multi-device 102 that need power to operate, in the manner described. The power supply 134 can further provider power to various components (e.g., a network device 136, a controller 132, and the like) of the body 130. The power supply 134 is one or more of one or more of batteries (e.g., lithium-polymer batteries), solar panels/cells, hydrogen cells, tethered power connections, combustion engines, power links (e.g., laser transmitter), or the like.

The multi-device system 100 (e.g., the body 130) includes a controller 132 configured to generate, transmit, or otherwise provide operational instructions to the devices 104a-104n, the tools 106a-106n, the movement mechanism 120, and other components of the multi-device 102 that need the operational instructions to operate, in the manner described. The controller 132 is further configured to control the movement of the body 130 (e.g., the robotic manipulator) to position the multi-device 102 relative to the subject. The controller 132 is further configured to receive and process the data collected by the devices 104a-104n for diagnosis or for generating additional operational instructions. The additional operational instructions include operational instructions corresponding to further movements of the body 130 (e.g., the robotic manipulator) to further position the multi-device 102 relative to the subject, operational instructions corresponding to further movement of the movement mechanism 120, and operational instructions for the devices 104a-104n and tools 106a-106n.

The controller 132 includes a processor and a memory for performing the functions described herein. The processor may be implemented as a general-purpose processor, an Application Specific Integrated Circuit (ASIC), one or more Field Programmable Gate Arrays (FPGAs), a Digital Signal Processor (DSP), a group of processing components, or other suitable electronic processing components. The memory (e.g., Random Access Memory (RAM), Read-Only Memory (ROM), Non-volatile RAM (NVRAM), Flash Memory, hard disk storage, etc.) stores data and/or computer code for facilitating at least some of the various processes described herein. The memory includes tangible, non-transient volatile memory, or non-volatile memory. In this regard, the memory stores programming logic that, when executed by the processor, controls the operations of the body 130 and the multi-device 102 in the manner described herein.

In some arrangements, a device (e.g., the device 104n) includes a force controller (e.g., a force controller 103) configured to detect the reactionary force that a surface (e.g., skin of a subject) exerts against the device as the device monitors, diagnoses, treats, or performs another suitable medical procedure on the subject. For example, the force controller 103 can be positioned behind or directly behind an activation slot of the device 104n. Alternatively, the force controller 103 can be positioned behind or directly behind an activation slot of the multi-device 102. The force controller 103 can be positioned between the device 104a and the body interface 110. A load cell can be positioned behind the activation slot for detecting an amount of reactionary force that a surface exerts against the activated device 104n as the activated device 104n exerts an opposite force against the surface. In an example in which the device 104n, when activated, utilizes physical contact against a subject for operation (e.g., an ultrasound imaging device or a Transcranial Doppler (TCD) device), the force controller 103 can detect an amount of force exerted by the surface against the device 104n. The data corresponding to the detected force can be sent by the force controller 103 to the first data communication interface 114, to be sent to the controller 132 for processing in the manner similar to described with respect to the data collected by the device 104n. Based on the detected force, the multi-device system 100 (e.g., the controller 132) can regulate the force that the activated device 104n exerts against the subject so that the device 104n is not causing pain to the subject or to ensure that the device 104n is pressed against the subject using an adequate amount of force so that the device 104n can adequately perform operations.

In some arrangements, the body interface 110 is configured to move relative to the body 130 to selectively activate at least one of the devices 104a-104n at a given time. In that regard, the movement mechanism 120 or another suitable movement mechanism can be configured to selectively activate at least one of the devices 104a-104n by moving the body interface 110 with respect to the body 130. The at least one of the devices 104a-104n is activated when the at least one of the devices 104a-104n is receiving the power and the operational instructions from the body 130.

In that regard, the body interface includes a first connection interface 112 operatively coupled to each of devices 104a-104n. The body 130 includes a second connection interface 140 operatively coupled to the power supply 134 and the controller 132. The second connection interface 140 is configured to provide the power and the operational instructions to the first connection interface 112 for the activated at least one of the devices 104a-104n by operatively coupling to the first connection interface 112 when the body interface 110 is moved (e.g., rotated) relative to the body to a predetermined position. The first connection interface 112 is configured to provide the data collected by the at least one of the devices 104a-104n to the second connection interface 140 by operatively coupling to the second connection interface 140.

In some examples as shown, the first connection interface 112 includes a first power interface 118 and a first data communication interface 114 separate from the first power interface 118. The second connection interface 140 includes a second power interface 144 and a second data communication interface 142 separate from the second power interface 144.

The power supply 134 is operatively coupled to the second power interface 144. The second power interface 144 is configured to operatively couple to the first power interface 118 when the body interface 110 is moved relative to the body 130 to the predetermined position to provide the power received from the power supply 134 to the first power interface 118. The second power interface 144 includes a suitable wire or cable that connects the power supply 134 to the end of the body 130 (e.g., at the second connection interface 140). The second power interface 144 includes a contact interface (e.g., a metal surface, a metal contact point, or the like). The first power interface 118 includes a contact interface corresponding to each of the devices 104a-104n.

When the body interface 110 (e.g., the first connection interface 112) is moved (e.g., rotated) to a first predetermined position relative to the end of the body 130 (e.g., the second connection interface 140), a first contact interface of the first power interface 118 that corresponds to the device 104a comes in contact with the contact interface of the second power interface 144. In other words, the first contact interface of the first power interface 118 and the contact interface of the second power interface 144 are aligned in the first predetermined position. Responsive to the contact and the alignment, the first contact interface of the first power interface 118 is receiving power from the contact interface of the second power interface 144.

When the body interface 110 (e.g., the first connection interface 112) is moved (e.g., rotated) to a second predetermined position relative to the end of the body 130 (e.g., the second connection interface 140), another contact interface of the first power interface 118 that corresponds to another device (e.g., the device 104b) comes in contact with the contact interface of the second power interface 144. In other words, the another contact interface of the first power interface 118 and the contact interface of the second power interface 144 are aligned in the second predetermined position. Responsive to the contact and the alignment, the another contact interface of the first power interface 118 is receiving power from the contact interface of the second power interface 144.

The controller 132 is operatively coupled to the second data communication interface 142. The second data communication interface 142 is configured to operatively couple to the first data communication interface 114 when the body interface 110 is moved relative to the body 130 to the predetermined position to provide the operational instructions to the first data communication interface 114 and to receive the data collected by the at least one of the devices 104a-104n from the first data communication interface 114. The second data communication interface 142 includes a suitable wire or cable that connects the controller 132 to the end of the body 130 (e.g., at the second connection interface 140). The second data communication interface 142 includes a contact interface (e.g., a metal surface, a metal contact point, or the like). The first data communication interface 114 includes a contact interface corresponding to each of the devices 104a-104n.

When the body interface 110 (e.g., the first connection interface 112) is moved (e.g., rotated) to a first predetermined position relative to the end of the body 130 (e.g., the second connection interface 140), a first contact interface of the first data communication interface 114 that corresponds to the device 104a comes in contact with the contact interface of the second data communication interface 142. In other words, the first contact interface of the first data communication interface 114 and the contact interface of the second data communication interface 142 are aligned in the first predetermined position. Responsive to the contact and the alignment, the first contact interface of the first data communication interface 114 is receiving operational instructions from the contact interface of the second data communication interface 142, and the first contact interface of the first data communication interface 114 is sending captured data to the contact interface of the second data communication interface 142.

When the body interface 110 (e.g., the first connection interface 112) is moved (e.g., rotated) to a second predetermined position relative to the end of the body 130 (e.g., the second connection interface 140), another contact interface of the first data communication interface 114 that corresponds to another device (e.g., the device 104b) comes in contact with the contact interface of the second data communication interface 142. In other words, the another contact interface of the first data communication interface 114 and the contact interface of the second data communication interface 142 are aligned in the second predetermined position. Responsive to the contact and the alignment, the another contact interface of the first data communication interface 114 is receiving power from the contact interface of the second data communication interface 142, and the another contact interface of the first data communication interface 114 is sending captured data to the contact interface of the second data communication interface 142.

In some examples, when the body interface 110 (e.g., the first connection interface 112) is moved (e.g., rotated) to a first predetermined position relative to the end of the body 130 (e.g., the second connection interface 140), a first contact interface of the first power interface 118 that corresponds to the device 104a comes in contact with the contact interface of the second power interface 144, and a first contact interface of the first data communication interface 114 that corresponds to the device 104a comes in contact with the contact interface of the second data communication interface 142. That is, when the body interface 110 is moved to a first predetermined position relative to the end of the body 130, the device 104a is receiving both power and operational instructions from the second data communication interface 142 and the second power interface 144. The device 104a is also sending captured data to the second data communication interface 142. Therefore, the device 104 is deemed to be activated when the body interface 110 is moved to the first predetermined position.

In some arrangements, when the body interface 110 is moved to a predetermined position relative to the end of the body 130, one of the devices 104a-104n is activated in the manner described.

In other arrangements, when the body interface 110 is moved to a predetermined position relative to the end of the body 130, two or more of the devices 104a-104n are simultaneously activated in the manner described. For example, instead of one contact, each of the second data communication interface 142 and the second power interface 144 may have multiple contacts disposed on the second connection interface 140. Multiple contacts corresponding to two or more of the devices 104a-104n are disposed on each of the first data communication interface 114 and the first power interface 118. When the body interface 110 is moved to the predetermined position relative to the end of the body 130, the multiple contacts of the second data communication interface 142 contacts and aligns with the multiple contacts of the first data communication interface 114, and the multiple contacts of the second power interface 144 contacts and aligns with the multiple contacts of the first power interface 118. This allows multiple ones of the devices 104a-104n to be simultaneously activated, to perform a joint task or to obtain data of the subject simultaneously.

In some arrangements, instead of two separate links (e.g., contacts) for power and data/operational instructions, the first connection interface 112 and the second connection interface 140 can transfer power and data/operational instructions via a same link. For example, the second connection interface 140 is configured to provide the power and the operational instructions to the first connection interface 112 by providing to the first connection interface 112 modulated power signals that include the power from the power supply 134 and the operational instructions from the controller 132. In that regard, the second connection interface 140 includes a modulator that modulates the operational instructions with the power. The first connection interface 112 includes a demodulator that demodulates the modulated power signals to separate the power signals from the operational instructions. The first connection interface 112 and the second connection interface 140 each includes a contact, such that when the contacts are in contact with and align one another, the modulated power signals are communicated from the second connection interface 140 to the first connection interface 112.

In some arrangements, instead of a physical contact connection, the first connection interface 112 and the second connection interface 140 can communicate power and data/operational instructions wirelessly. With respect to power, each of the first power interface 118 and the second power interface 144 includes a loop or coil configured to communicate power wirelessly via induction. With respect to data/operational instructions, each of the first data communication interface 114 and the second data communication interface 142 includes suitable network device configured to communicate wireless signals via Bluetooth®, Wi-Fi, cellular, or another suitable wireless network. In the implementations in which modulated power signals are implemented, the first connection interface 112 and the second connection interface 140 each includes a loop or coil configured to communicate the modulated power signals via induction.

In some arrangements, the multi-device 102 may include its own power supply (e.g., a rechargeable battery) such that any electrical connection between the body 130 and the multi-device 102 is not necessary.

In some arrangements, the body interface 110 includes a locking device 122 configured to structurally maintain a predetermine position to maintain connection. The locking device 122 may include one or more of magnets, spring-loaded pins, clamps, locks, latches, bolts, buckles, clips, hook-and-look fasteners, pegs, pins, rivets, snap fasteners, straps, and another suitable mechanical fastener. Alternatively or additionally, another locking device (such as but not limited to, the locking device 122) can be disposed on the body 130 (e.g., on the second connection interface 140). In one example, the contact interfaces described herein can include magnets, or magnets are disposed in or around the contact interfaces such that magnetic force maintains the relative positions of the body and the body interface 110. In that regard, the magnets for a contact interface in the body interface 110 have the opposite polarity as compared to the magnets for a contact interface in the body 130.

In some arrangements, the second data communication interface 144 may include data transmission component (e.g., a fiber optics ring) configured to receive data from the activated ones of the devices 104a-104n. The first data communication interface 114 includes a component corresponding to each of the devices 104a-104n. The component can fire the collected data onto the fiber optics ring. The fiber optics ring can then relay the data to the controller 132. This enables one or more activated ones of the devices 104a-104n to send data to the second data communication interface 144 regardless of a position of the body interface 110 relative to the body 130. This further enables multiple activated devices to send data to the second data communication interface 144 simultaneously.

Any number of combination of the examples described herein relating to transfer of power, operational instructions, and data can be implemented by the body interface 110 (the first connection interface 112) and the second connection interface 140.

In some arrangements, a device (e.g., the device 104n) includes a force controller (e.g., a force controller 103) configured to detect the reactionary force that a surface (e.g., skin of a subject) exerts against the device as the device monitors, diagnoses, treats, or performs another suitable medical procedure on the subject. For example, the force controller 103 can be positioned behind or directly behind an activation slot of the device 104n. Alternatively, the force controller 103 can be positioned behind or directly behind an activation slot of the multi-device 102. The force controller 103 can be positioned between the device 104a and the body interface 110. A load cell can be positioned behind the activation slot for detecting an amount of reactionary force that a surface exerts against the activated device 104n as the activated device 104n exerts an opposite force against the surface. In an example in which the device 104n, when activated, utilizes physical contact against a subject for operation (e.g., an ultrasound imaging device or a Transcranial Doppler (TCD) device), the force controller 103 can detect an amount of force exerted by the surface against the device 104n. The data corresponding to the detected force can be sent by the force controller 103 to the first data communication interface 114, to be sent to the controller 132 for processing in the manner similar to described with respect to the data collected by the device 104n. Based on the detected force, the multi-device system 100 (e.g., the controller 132) can regulate the force that the activated device 104n exerts against the subject so that the device 104n is not causing pain to the subject or to ensure that the device 104n is pressed against the subject using an adequate amount of force so that the device 104n can adequately perform operations.

In some arrangements, the body 130 includes a network device 136 configured to receive data from another computing platform and/or send data to another computing platform. For example, the network device 136 can be configured to receive the operational instructions from a computing platform, which includes a user interface configured to receive the operational instructions or instructions to control the movement of the body 130 (e.g., the robotic manipulator) from a healthcare provider. The network device 136 can be further configured to send the data collected by the devices 104a-104n to the computing platform. The computing platform can process the data for diagnosis or for generating additional operational instructions. The network device 136 includes at least one transceiver that performs transmit/receive functions via Bluetooth®, Wi-Fi, cellular, or another suitable wireless network. The network device 136 can include separate transmit and receive circuitries, or can include a transceiver that combines transmitter and receiver functions.

In some arrangements, the body 130 includes a mounting mechanism 138 configured to detachably mount the multi-device system 100 (e.g., the body 130) to an object. For example, the mounting mechanism 138 can be configured to anchor the body 130 on a surface (e.g., a table, a bed, a gurney, a stretcher, a workbench, a wall, a vehicle, a computing platform or workstation, and the like). The mounting mechanism 138 is disposed on an end of the body 130 (e.g., a robotic manipulator) that is opposite to the end on which the second connection interface 140 is disposed. The mounting mechanism 138 is a mechanical fastener, examples of which include but are not limited to, magnets, spring-loaded pins, clamps, locks, latches, bolts, buckles, clips, hook-and-look fasteners, pegs, pins, rivets, snap fasteners, straps, strings, and another suitable mechanical fastener.

In some arrangements, the body 130 includes a semi-automatic switch 148 and a manual interface 150. The manual interface 150 configured to allow the multi-device 102 to be moved relative to the subject by a user (e.g., a healthcare provider) of the multi-device system 100. In that regard, the manual interface 150 is disposed on one or more of the multi-device 102 or the body 130 to allow the user to move the multi-device 102 and/or the body 130 by moving (e.g., pulling, pushing, dragging, and the like) the manual interface 150. Examples of the manual interface 150 include but are not limited to, a handle, a knob, a rope, a groove, a grip, and the like.

The semi-automatic switch 148 being in a first state corresponds to allowing the multi-device 102 and/or the body 130 to be moved relative to the subject by the user via the manual interface 150. The semi-automatic switch 148 being in a second state corresponds to disallowing the multi-device 102 and/or the body 130 to be moved relative to the subject by the user. The semi-automatic switch 148 can be a type of switch connected to the controller 132. Responsive to determining that the semi-automatic switch 148 is in the first state, the controller 132 enables the user to move the multi-device 102 by loosening one or more joints on the body 130 (e.g., the robotic manipulator), such that the user can move the manual interface 150 (and therefore the multi-device 102 and/or the body 130) to a designed position. Responsive to determining that the semi-automatic switch 148 is in the second state, the controller 132 disallows the movement of the multi-device 102 and/or the body 130 by tightening one or more joints on the body 130 (e.g., the robotic manipulator), such that the body 130 and the multi-device 102 are rigid and unresponsive to user manipulation.

In some arrangements, the tools 106a-106n can be selectively coupled to one or more of the devices 104a-104n to enhance or change data characteristics of the one or more of the devices 104a-104n. While the multi-device 102 is shown to include the tools 106a-106n, one of ordinary skill in the art can appreciate that the multi-device 102 may have any number of tools disposed the multi-device 102. The tools 106a-106n are configured to move (e.g., rotate) relative to the devices 104a-104n to selectively couple to the one or more of the devices 104a-104n. For example, the tools 106a-106n are fixed, attached, mounted, or otherwise disposed on a platform 124 (e.g., a disk). The tools 106a-106n can rotate with the platform 124. The platform 124 is configured to move (e.g., rotate) with respect to the body interface 110 on which the devices 104a-104n are disposed. The movement of the platform 124 causes a tool (e.g., the tool 106a) to become operatively coupled to (e.g., by aligning with) a device (e.g., the device 104a), such that the tool can enhance or change the data characteristics of the device. While FIG. 1 shows that each of the devices 104a-104n has a respective one of the tools 106a-106n operatively coupled thereto, one of ordinary skill in the art can appreciate that any number of tools can be operatively coupled to any number of devices at any time.

In some examples, the tool 106a (e.g., a first lens) is configured to operatively couple to the device 104a (e.g., a camera) when the tools 106a-106n (e.g., and the platform 124) are moved to a first relative position relative to the devices 104a-104n (and the body interface 110). The tool 106a being operatively coupled to the device 104a enables the device 104a to output first data (e.g., a first type of data such as but not limited to, an image with a first resolution). The tool 106b (e.g., a second lens) is configured to operatively couple to the device 104a when the tools 106a-106n (e.g., and the platform 124) are moved to a second relative position relative to the devices 104a-104n (and the body interface 110). The tool 106b being operatively coupled to the device 104a enables the one of the devices 104a to output second data different from first data. The second data may be a second type of data such as but not limited to, an image with a second resolution.

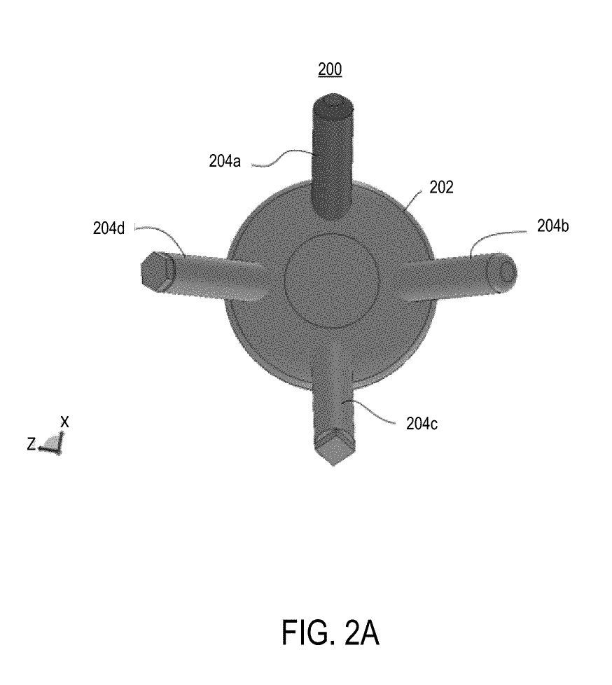

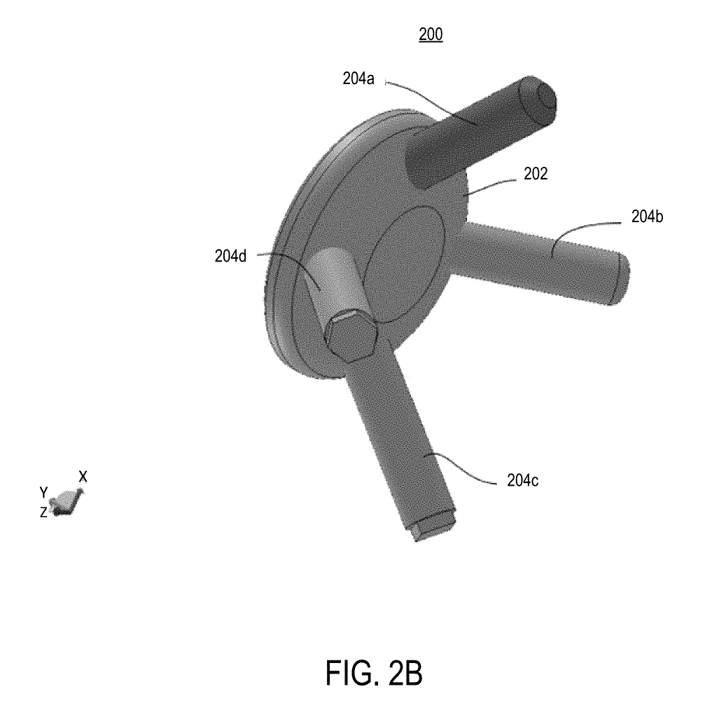

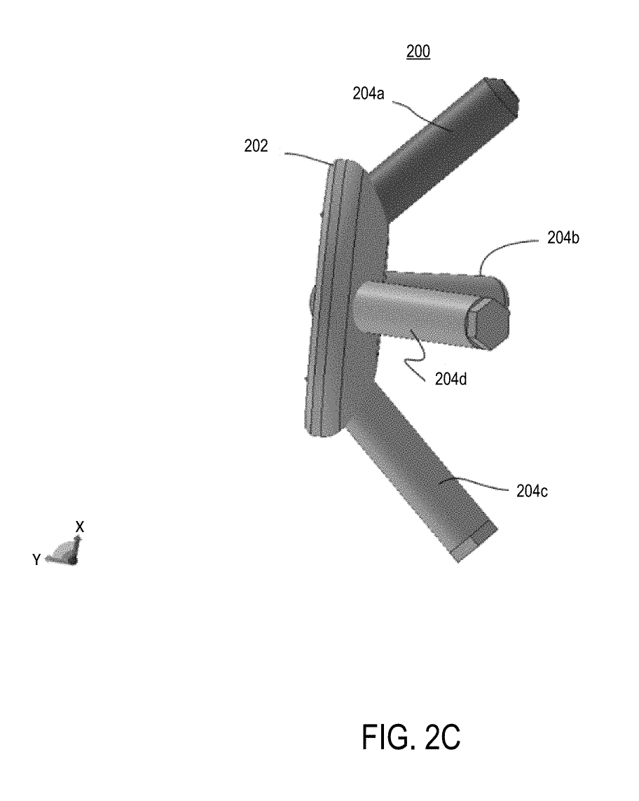

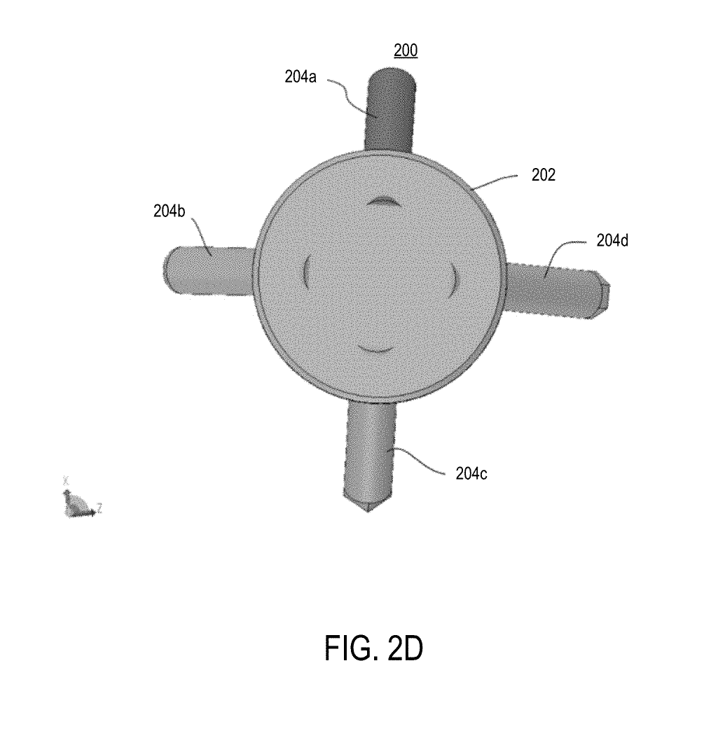

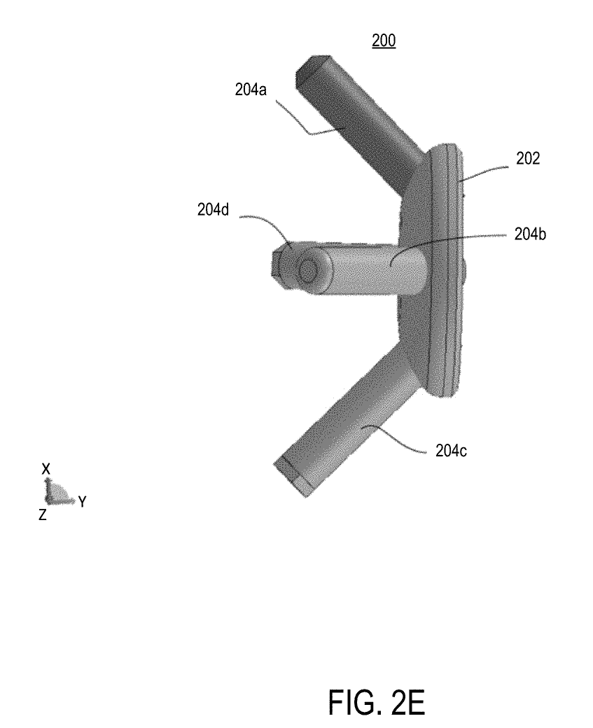

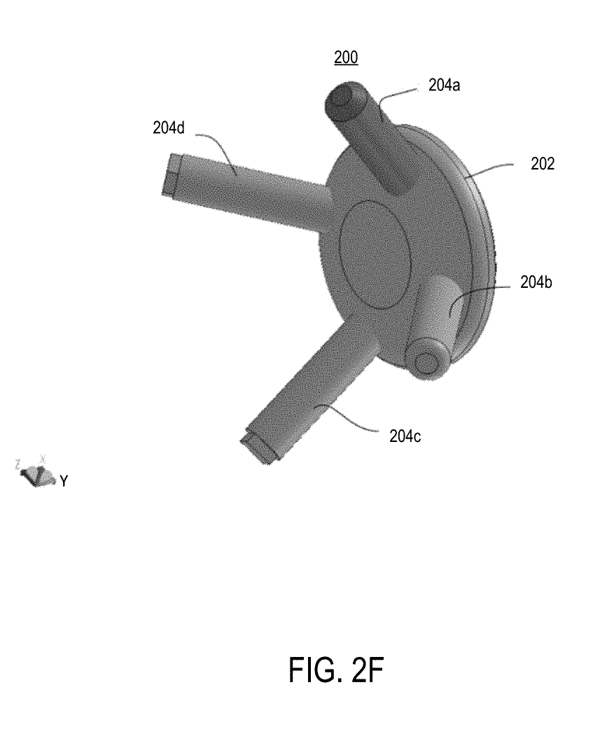

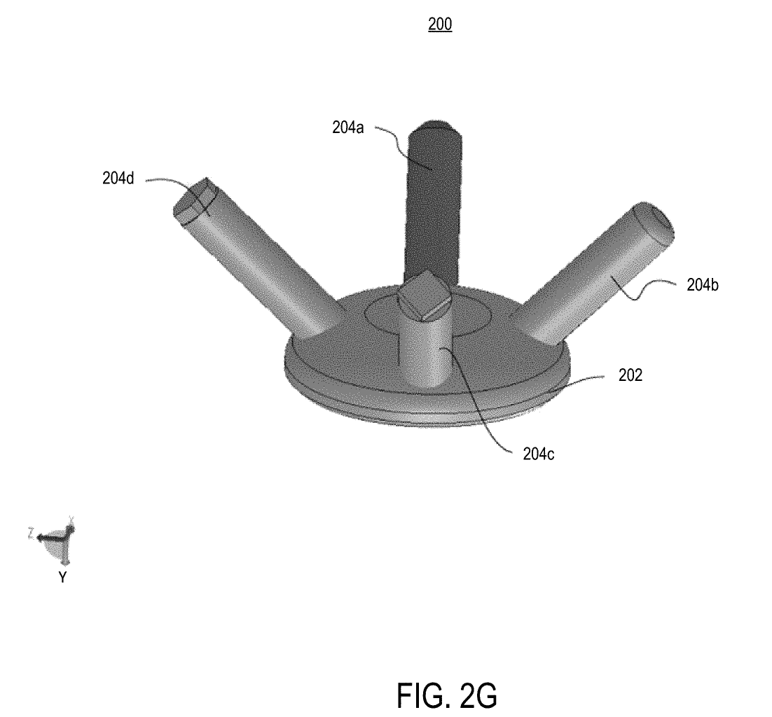

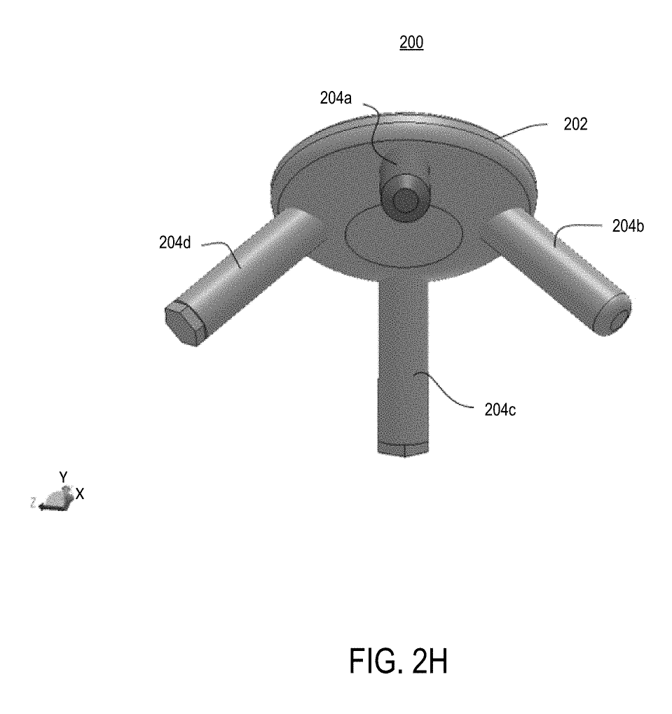

FIG. 2A illustrates a front view of a multi-device 200 according to various arrangements. FIG. 2B illustrates a perspective view of the multi-device 200 shown in FIG. 2A according to various arrangements. FIG. 2C illustrates a side view of the multi-device 200 shown in FIG. 2A according to various arrangements. FIG. 2D illustrates a rear view of the multi-device 200 shown in FIG. 2A according to various arrangements. FIG. 2E illustrates a side view of the multi-device 200 shown in FIG. 2A according to various arrangements. FIG. 2F illustrates a perspective view of the multi-device 200 shown in FIG. 2A according to various arrangements. FIG. 2G illustrates a perspective view of the multi-device 200 shown in FIG. 2A according to various arrangements. FIG. 2H illustrates a perspective view of the multi-device 200 shown in FIG. 2A according to various arrangements.

Referring to FIGS. 1-2H, the multi-device 200 includes a body interface 202 and a plurality of devices 204a-204d. The multi-device 200 is an example implementation of the multi-device 102. The body interface 202 is an example implementation of the body interface 110. The devices 204a-204d are example implementations of the devices 104a-104n.

As shown, the body interface 202 is shaped as a hemisphere. In other examples, the body interface 220 has another suitable shape such as but not limited to, a cube, a cylinder, a torus, an ellipsoid, a disk (e.g., a parabolic disk), and the like. As shown, each of the devices 204a-204d are angled outward from a center of the body interface 202. Each of the devices 204a-204d are located at an outer perimeter of the hemispherical body interface 202. In other examples, the devices 204a-204d can be angled inward toward a focal point (e.g., in the example in which the body interface 202 is shaped as a parabolic disk, the devices 204a-204d can be angled toward a focal point of the parabolic disk).

In some arrangements, the body interface 202 is modular such that the body interface 202 can be attached to and detached from another body or instruments (e.g., the body 130, which can be a robot that provides power, operational instructions, and/or physical positioning to the devices 204a-204d with respect to a subject). In some arrangements, the body interface 202 is configured to rotate when attached to a body or an instrument (e.g., the body 130) such that the devices 204a-204d rotate with respect to the attached body, therefore toggling activation among the devices 204a-204d. For example, the body interface 202 can be configured to rotate about an axis through the center of the body interface 202. The axis is perpendicular to a surface of the body interface 202 on which the body is attached in some examples. In the examples in which the body interface 202 has the parabolic disk shape, the axis is through the focal point of the parabolic disk. Further details regarding the instrument and rotation of the devices 204a-204d is described herein.

As described, in some arrangements, each of the devices 204a-204d includes a device that is capable of providing healthcare to a subject (e.g., imaging, diagnosis, or treatment of the subject). In one example, one or more of the devices 204a, 204b, 204c, 204d include a probe. In one example, one or more of the devices 204a-204d include an imaging device configured to determine physiological characteristics of a subject (e.g., blood flow within a particular portion of the subject's body, skeletal composition of the subject, and so on). In one example, examples of each of the devices 204a-204d include but are not limited to, a camera, a therapy device (e.g., a laser, a syringe, an ablation device, a light therapy device, an electric shock therapy device, a stitching device (such as a surgical suture), a scalpel, a catheter, an insulin pump, a bandaging device, a respirator, and the like), an ultrasound range device, an ultrasound imaging device, a thermal imaging device, an endoscopy device, an electrography device, a tactile imaging device, a near-infrared imaging device, a near-infrared spectroscopy device, a nuclear medicine device, an optical camera device, a lighting device (e.g., for illumination), a micro-needle drug delivery device, a spectrometer device, a microphone device, an electroencephalography device, a metal detector, and the like.

In one example, one of the devices 204a-204d includes a near-infrared imaging probe that is configured to emit near-infrared light at the skin of a subject, and the near-infrared light that contacts the skin of the subject can distinctly reveal veins of the subject for quick identification by a healthcare provider. In one example, one of the devices 204a-204d includes a probe having a concave surface that is configured to be adjacent to or contact a scanning surface (e.g., a head of a subject). The concave surface is configured with a particular pitch to focus generated energy towards the scanning surface (e.g., a Transcranial Doppler (TCD) probe that is configured to be adjacent to or contact and align along a human head, while the TCD probe is configured to provide ultrasound wave emissions directed into the human head).

Further disclosure regarding probe systems that can be used in conjunction with the multi-device 200 can be found in non-provisional patent application Ser. No. 15/399,648, titled ROBOTIC SYSTEMS FOR CONTROL OF AN ULTRASONIC PROBE, and filed on Jan. 5, 2017, which is incorporated herein by reference in its entirety.

In one example, one of the devices 204a-204d includes a metal detector configured to be used to determine whether the subject has any metal objects (e.g., fillings, braces, or screws) that may interfere with operations of other devices or cause harm to the subject during the scan. In some examples, the metal detector is configured to detect and locate metal objects (e.g., bullets, fragments, or other foreign metal objects) in the subject.

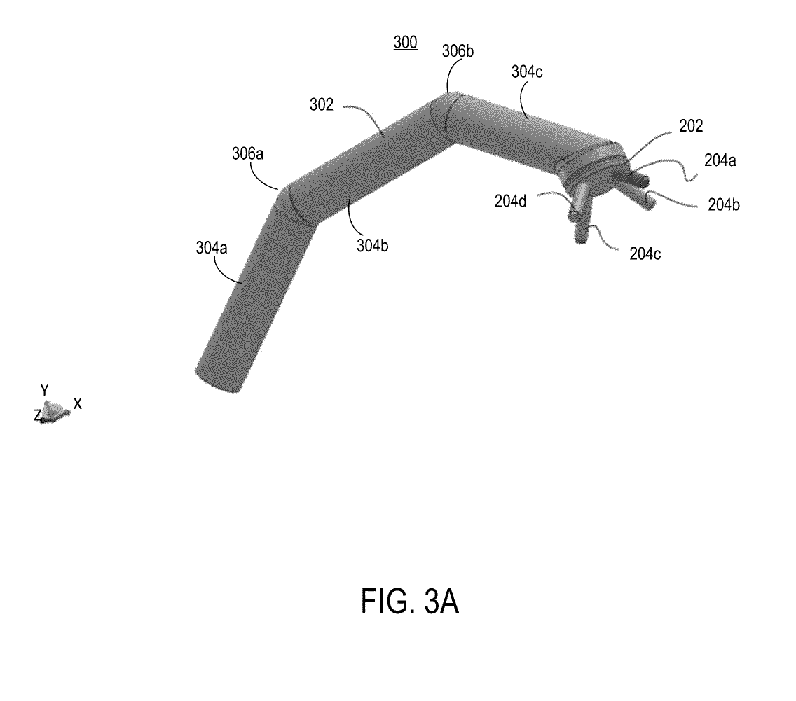

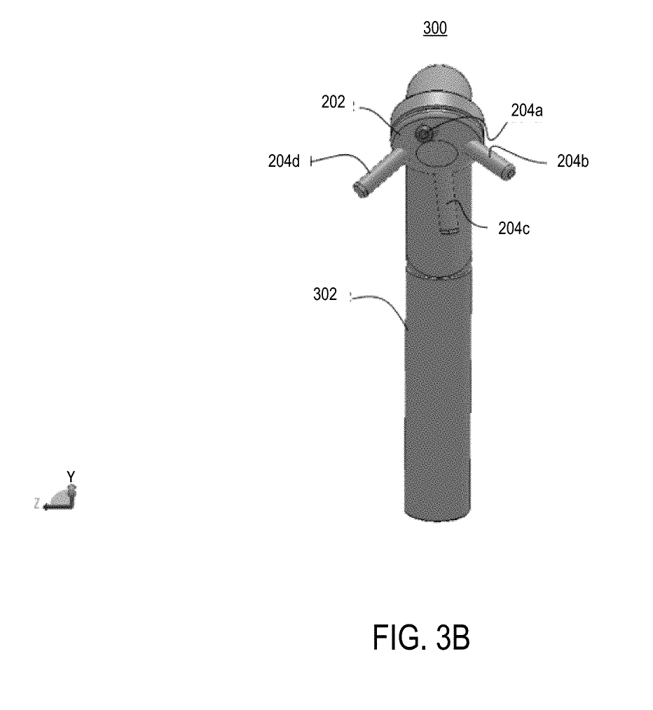

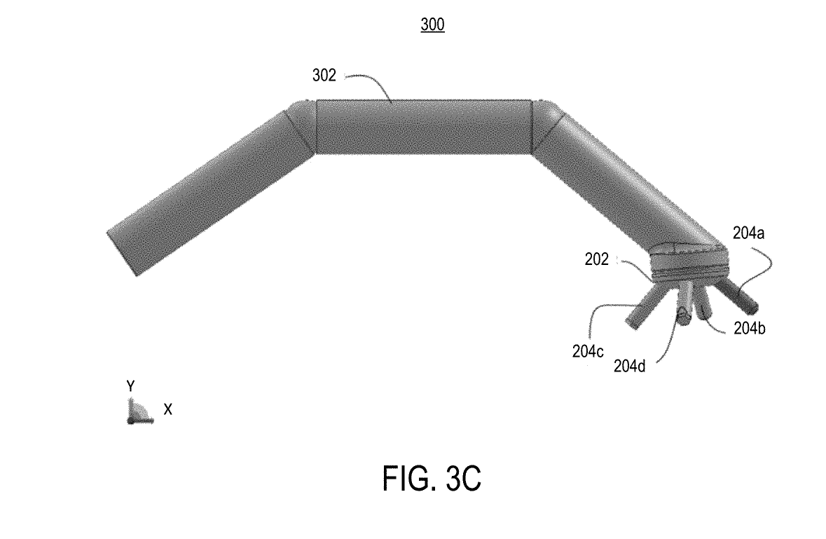

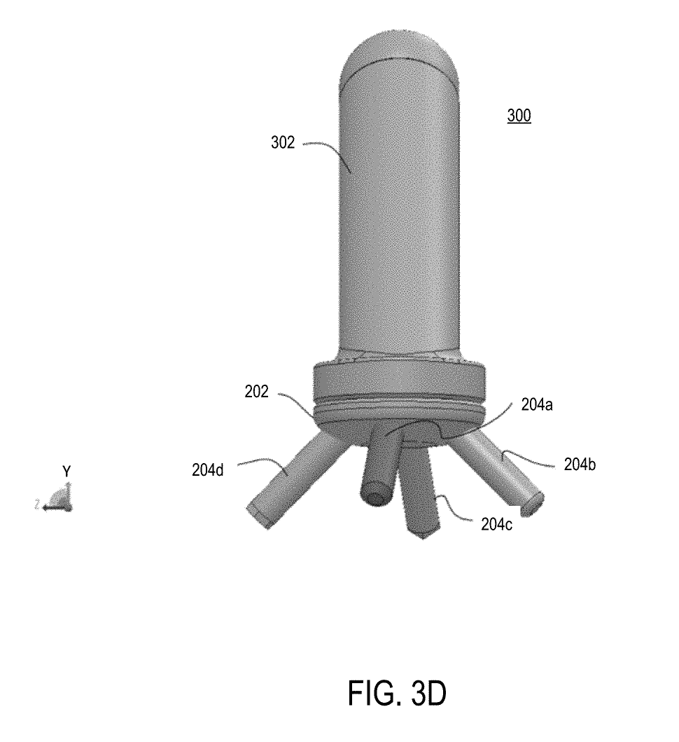

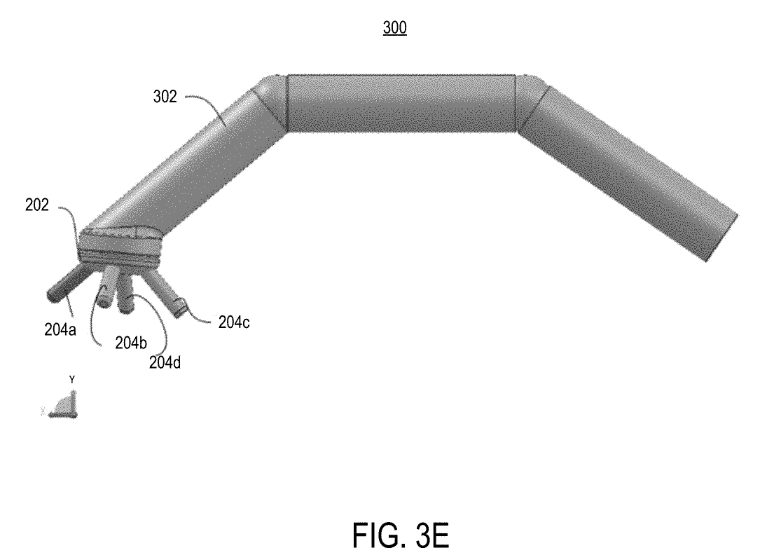

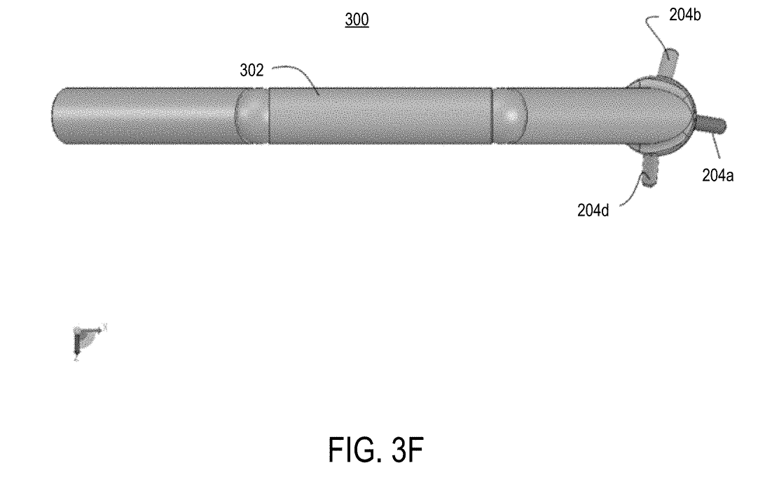

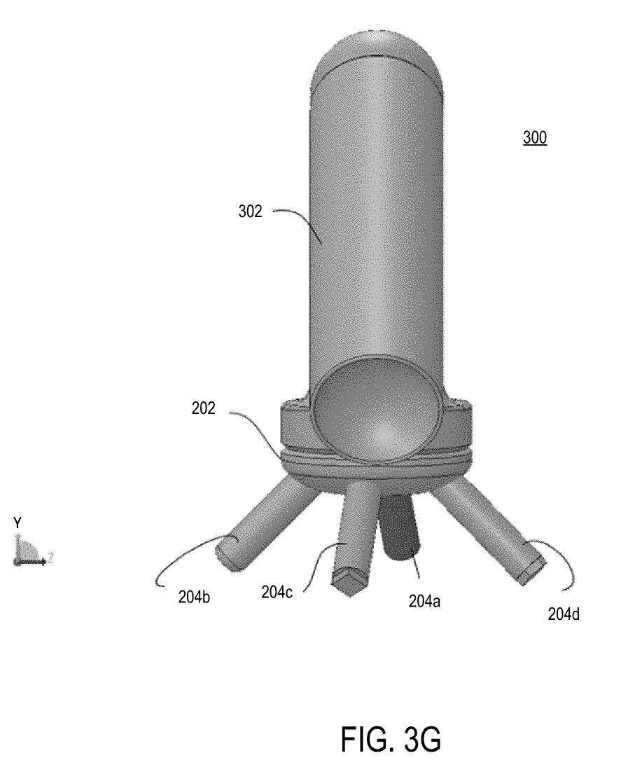

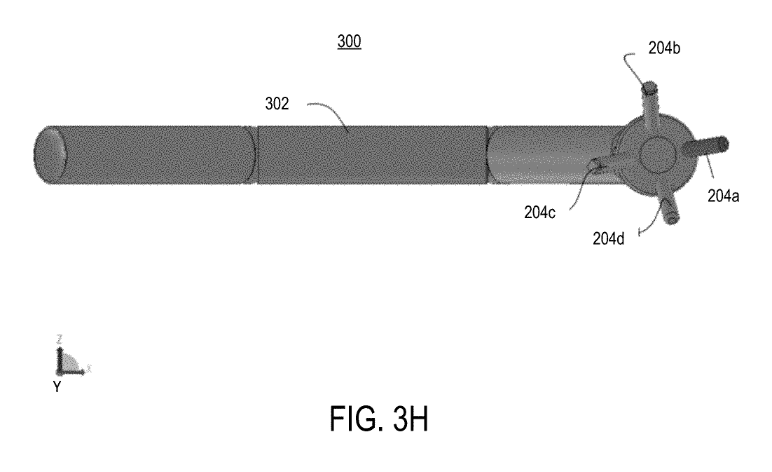

FIG. 3A illustrates a perspective view of a multi-device system 300 according to various arrangements. FIG. 3B illustrates a front view of the multi-device system 300 shown in FIG. 3A according to various arrangements. FIG. 3C illustrates a side view of the multi-device system 300 shown in FIG. 3A according to various arrangements. FIG. 3D illustrates a top view of the multi-device system 300 shown in FIG. 3A according to various arrangements. FIG. 3E illustrates a side view of the multi-device system 300 shown in FIG. 3A according to various arrangements. FIG. 3F illustrates an overhead view of the multi-device system 300 shown in FIG. 3A according to various arrangements. FIG. 3G illustrates a rear view of the multi-device system 300 shown in FIG. 3A according to various arrangements. FIG. 3H illustrates a bottom view of the multi-device system 300 shown in FIG. 3A according to various arrangements.

In some arrangements, the multi-device system 300 includes the multi-device 200 attached to a body 302. The body 302 corresponds to the body 130. In some arrangements, the body 302 includes a robot, robotic manipulator, robotic arm, or the like that automatically operates each of the devices 204a-204d, including maneuvering the devices 204a-204d at a correct and optimal position along the body of a subject. In that regard, the body 302 includes arm segments 304a-304c connected by joints 306a and 306b as shown. A control circuit (e.g., the controller 132, the computing platform configured to send the instructions via a network to the body 130, and the like) is configured to control the movements of the arm segments 304a-304c and the joints 306a and 306b to position the multi-device 200 with respect to the subject (e.g., along, close to, or contacting the body of the subject) in the manner described. In some examples, at least one of the joints 306a and 306b has six DOFs, such that the body 302 can also be referred to as a redundant manipulator. In other arrangements, at least one of the joints 306a and 306b has fewer DOFs, such as five (e.g., the Cartesian coordinates (x, y, z), pan, and tilt) or is underactuated such that at least one of the joints 306a and 306b is controlled in part by a spring. Thus, the body 302 can be employed to move the devices 204a-204d to any appropriate location of a subject.

In some arrangements, the body 302 can be mounted (e.g., via the mounting mechanism 138) with a portable workstation that includes a monitor (e.g., for providing results of the operations performed on a subject by the multi-device system 300).

In addition, the body 302 can automatically select or selectively activate one or more of the devices 204a-204d for use. In some arrangements, the multi-device 200 activates one (e.g., the device 204a) of the devices 204a-204d at a time for use (the active device is shown as device 204a), such that the activated device 204a is angled towards a subject, while other inactive devices (e.g., the devices 204b, 204c, and 204d) are angled away from the subject. In some arrangements, the activated device 204a is aligned with and internally connected to components within the body 302 communicating power, operational instructions, and collected data, while the inactive devices 204b, 204c, and 204d are not connected or aligned as such. In some arrangements, to activate at least one of the inactive devices 204b, 204c, and 204d and to deactivate the active device 204a, the body 302 moves (e.g., rotates) the multi-device 200 such that the active device 204a is no longer in communication with internal electronics of the body 302, while another one of the devices 204b, 204c, and 204d is rotatably positioned to be connected such that the newly activated device is capable of operation with respect to a subject. Although four devices 204a-204d are shown in the figures, any desired number of different devices can be implemented as part of the multi-device system 300, such as, but not limited to, one, two, three, or more than four different devices.

In some arrangements, the multi-device system 300 is capable of atomic force microscopy in conjunction with utilization of the devices 204a-204d (or one of the devices 204a-204d can include an atomic force microscope). For example, the multi-device 200 (e.g., the device 204a) can image a body part of a subject using atomic force microscopy, applying pressure along the body of the subject in incremental distances to generate a 3D contour of the subject. The body 302 can then operate one of the devices 204a-204d using the imaged body part. That is, after imaging the body part, the body 302 positions one of the devices 204a-204d at an optimal position along the body part using the image of the body part for operation of the one of the devices 204a-204d, such as for determining cerebral blood flow velocity in a subject's head using a TCD probe). In some arrangements, the multi-device system 300 can monitor a volume of a body part of a subject over time to determine whether the volume of the body part changes. For example, the multi-device system 300 can detect that a volume of a subject's arm has increased over the span of a few weeks, which can lead to a further diagnosis regarding the cause of the volume increase (e.g., swelling of joints in the arm).

Accordingly, in some arrangements, the multi-device system 300 includes an automatic system for imaging, diagnosing, and treating a subject. In an example in which a subject is positioned near the multi-device system 300, the multi-device system 300 can automatically image the subject's body volume, utilize the devices 204a-204d with respect to the subject's body based on the image, and provide results of scans made by the devices 204a-204d in the manner described. Accordingly, a qualified healthcare provider or technician is not necessary for operating the multi-device system 300, as the operation can be automated or at least semi-automated.

As an example, a subject is positioned near the multi-device system 300. Upon initiation of operation, the multi-device system 300 selects the device 204a, which is a camera for providing a coarse three-dimensional image of the subject (e.g., via atomic force microscopy or ultrasound imaging). Once the coarse three-dimensional model of the subject is obtained, the multi-device 300 selects the device 204b (e.g., by rotating the device 204b into the activation slot such that the device 204a becomes deactivated and the device 204b becomes activated) that is a high-definition camera for obtaining a fine three-dimensional model of the subject (e.g., via atomic force microscopy or ultrasound imaging). Once the fine three-dimensional image of the subject is obtained, the multi-device system 300 selects another device for performing any desired diagnostic operations on the subject. For example, the multi-device system 300 can select the device 204c that is a high-resolution imager that scans the skin of the subject and identifies any abnormal skin conditions that may be pre-cancerous or cancerous (e.g., the device 204c can be a high-resolution camera that identifies any discolorations of the skin from the skin's default color tone). As a further example, the multi-device system 300 can select the device 204d that is a thermal imaging device and scan the locations of the skin discolorations to confirm via thermal imaging whether those locations are pre-cancerous or cancerous.

The above example is merely illustrative of the capabilities of the multi-device system 300, as various sequencing of imaging and diagnostic operations and scans can be utilized to provide healthcare, depending on the scenario and the subject. In some arrangements, the multi-device system 300 is located in hospitals and in physician offices such that it can be implemented as part of a routine physical examination. In some arrangements, the multi-device 300 is located in ambulances as part of emergency healthcare services.

In other arrangements, the multi-device system 300 is semi-automatic or manual such that a healthcare provider manually selects (e.g., rotates) a device 204a, 204b, 204c, or 204d for activation, maneuvers the multi-device system 300 around a subject to appropriately position the device 204a, 204b, 204c, or 204d, and manually initiates use of the device 204a, 204b, 204c, or 204d.

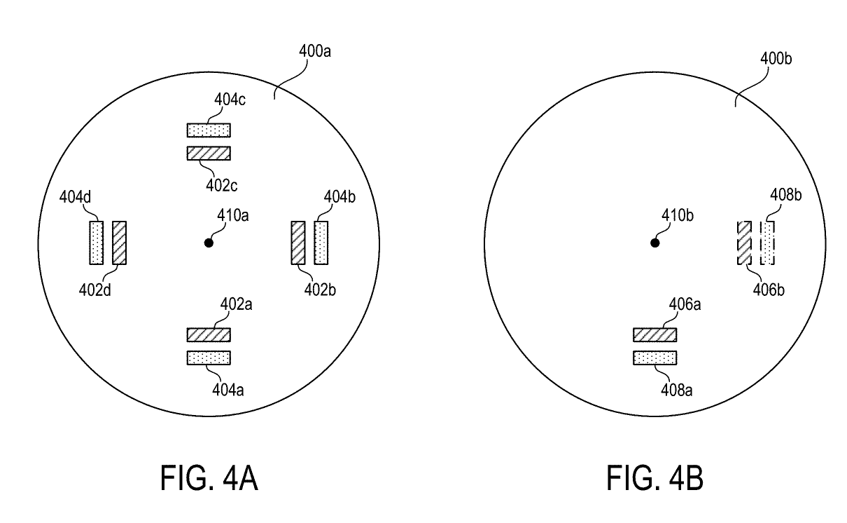

FIG. 4A illustrates a first connection interface 400a of a body interface (e.g., the body interface 110 of FIG. 1) of a multi-device (e.g., the multi-device 102 of FIG. 1) according to various arrangements. FIG. 4B illustrates a second connection interface 400b of a body (e.g., the body 130 of FIG. 1) according to various arrangements. Referring to FIGS. 1-4B, contact interfaces 402a-402d, 404a-404d, 406a, 406b, 408a, and 408b are example implementations of the contact interfaces described with reference to FIG. 1. The first connection interface 400a and the second connection interface 400b are example implementations of the first connection interface 112 and the second connection interface 140, respectively.

The second connection interface 400b includes at least a contact interface 406a that is operatively connected to the power supply 134 and is configured to provide power to one of the devices (e.g., the devices 204a-204d) disposed on the body interface on which the first connection interface 400a is positioned. The second connection interface 400b further includes at least a contact interface 408a that is operatively connected to the controller 132 and is configured to provide operational instructions to one of the devices (e.g., the devices 204a-204d) disposed on the body interface and receive data collected by the one of the devices. Therefore, the contact interfaces 406a and 408a are two separate contact interfaces spaced apart from one another with a gap. The combination of the contact interfaces 406a and 408a provides power and data/operational instructions communication capabilities, to activate one of the devices. In that regard, the contact interfaces 406a and 408a correspond to an activation slot to activate one of the devices. The second connection interface 400b may also have one or more additional activation slots (e.g., an additional activation slot for the contact interfaces 406b and 408b) configured to provide power and data/operational instructions communication capabilities to activate one or more additional devices, to enable simultaneous operation of two or more devices. The contact interfaces 406b and 408b are contact interfaces such as but not limited to, the contact interfaces 406a and 408a. While a given activation slot is shown to include two separate contact interfaces 406a and 406a for power and data/operational instructions, separately, in alternative arrangements, a given activation slot may have a single contact interface configured to provide power and data/operational instructions, for example, through modulated power signals as described herein.

The first connection interface 400a includes at least a contact interface 402a that is operatively connected to a first device (e.g., the device 204a) and is configured to provide power to the first device disposed on the body interface on which the first connection interface 400a is located. The first connection interface 400a further includes at least a contact interface 404a that is operatively connected to a controller (e.g., a microprocessor or another suitable processing unit) for the first device (e.g., the device 204a) and is configured to communicate data/operational instructions to and from the first device. Therefore, the contact interfaces 402a and 404a are two separate contact interfaces spaced apart from one another with a gap. The combination of the contact interfaces 402a and 404a provide power and data/operational instructions communication capabilities when the contact interfaces 402a and 404a align with and contact the contact interfaces 406a and 408a (or the contact interfaces 406b and 408b), respectively, to activate the first device.

Similarly, the combination of the contact interfaces 402b and 404b provide power and data/operational instructions communication capabilities when the contact interfaces 402b and 404b align with and contact the contact interfaces 406a and 408a (or the contact interfaces 406b and 408b), respectively, to activate a second device (e.g., the device 204b). The combination of the contact interfaces 402c and 404c provide power and data/operational instructions communication capabilities when the contact interfaces 402c and 404c align with and contact the contact interfaces 406a and 408a (or the contact interfaces 406b and 408b), respectively, to activate a third device (e.g., the device 204c). The combination of the contact interfaces 402d and 404d provide power and data/operational instructions communication capabilities when the contact interfaces 402d and 404d align with and contact the contact interfaces 406a and 408a (or the contact interfaces 406b and 408b), respectively, to activate a fourth device (e.g., the device 204d).

One or more of the first connection interface 400a and the second connection interface 400b can be configured to be rotated with respect to one another, to align the contact interfaces (e.g., the contact interfaces 402a and 404a) with contact interfaces 406a and 408a, to activate a device (e.g., the first device). As described, a movement mechanism (e.g., the movement mechanism 120) on one or both of the body interface (e.g., the body interface 110) and an end of the body 130 is configured to rotate the first connection interface 400a and/or the second connection interface 400b about centers 410a and 410b. Assembling the first connection interface 400a and the second connection interface 400b includes rotatably supporting the first connection interface 400a and the second connection interface 400b such that the centers 410a and 420a are aligned. As shown, the first connection interface 400a and the second connection interface 400b have a same shape (e.g., a circular surface). The contact interfaces 402a-402d, 404a-404d, 406a, 406b, 408a, and 408b can be rotated about an axis through the centers 410a and 410b. The axis can be perpendicular to the surfaces of the first connection interface 400a and the second connection interface 400b.

In particular, the contact interface 406a (and the contact interface 406b, if provided) is on a circular path defined by the contact interfaces 402a-402d, such that one of the contact interfaces 402a-402d can be aligned with the contact interface 406a (or the contact interface 406b, if provided). The contact interface 408a (and the contact interface 408b, if provided) is on a circular path defined by the contact interfaces 404a-404d, such that one of the contact interfaces 404a-404d can be aligned with the contact interface 408a (or the contact interface 408b, if provided). Although the contact interfaces 402a-402d and 404a-404d are spaced apart along respective circular paths, one of ordinary skill in the art can appreciate that other arrangements of the contact interfaces 402a-402d and 404a-404d can be suitably implemented.

If the contact interfaces 406b and 408b are provided, the relative positions of the contact interfaces 406a, 406b, 408a, and 408b correspond to the relative positions of two sets of the contact interfaces (e.g., the contact interfaces 402a, 402d, 404a, 404d) on the first connection interface 400a. This enables two sets of the contact interfaces to align and contact the contact interfaces 406a, 406b, 408a, and 408b simultaneously, for simultaneous activation and operation.

Responsive to the first connection interface 400a being in a first predetermined (relative) position relative to the second connection interface 400b, the contact interfaces 402a and 404a are aligned with and contact the contact interfaces 406a and 408a, respectively, to activate the first device. If the contact interfaces 406b and 408b are provided, in the first predetermined position, the contact interfaces 402d and 404d are aligned with and contact the contact interfaces 406b and 408b, respectively, to activate the fourth device. Responsive to the first connection interface 400a being in a second predetermined (relative) position relative to the second connection interface 400b, the contact interfaces 402b and 404b are aligned with and contact the contact interfaces 406a and 408a, respectively, to activate the second device. Responsive to the first connection interface 400a being in a third predetermined (relative) position relative to the second connection interface 400b, the contact interfaces 402c and 404c are aligned with and contact the contact interfaces 406a and 408a, respectively, to activate the third device. Responsive to the first connection interface 400a being in a fourth predetermined (relative) position relative to the second connection interface 400b, the contact interfaces 402d and 404d are aligned with and contact the contact interfaces 406a and 408a, respectively, to activate the fourth device.

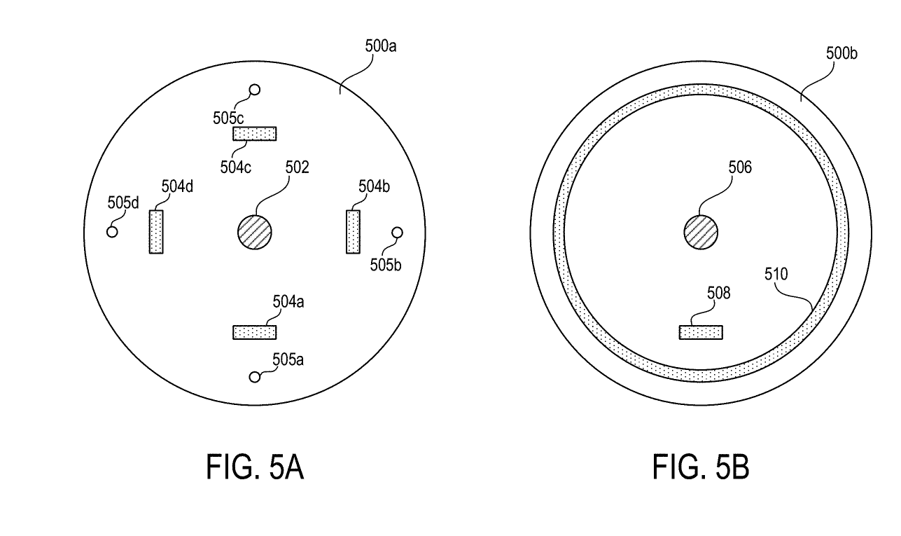

FIG. 5A illustrates a first connection interface 500a of a body interface (e.g., the body interface 110 of FIG. 1) of a multi-device (e.g., the multi-device 102 of FIG. 1) according to various arrangements. FIG. 5A illustrates a second connection interface 500b of a body (e.g., the body 130 of FIG. 1) according to various arrangements.

Referring to FIGS. 1-5B, the second connection interface 500b includes at least a contact interface 506 that is operatively connected to the power supply 134 and is configured to provide power to the devices (e.g., the devices 204a-204d) disposed on the body interface on which the first connection interface 500a is positioned. The second connection interface 500b further includes at least a contact interface 508 that is operatively connected to the controller 132 and is configured to provide operational instructions to one of the devices (e.g., the devices 204a-204d) disposed on the body interface. The combination of the contact interfaces 506 and 508 provide power and operational instructions communication capabilities, to activate one of the devices.

The first connection interface 500a includes at least a contact interface 502 that is operatively connected to devices (e.g., the devices 204a-204d) and is configured to provide power to all the devices disposed on the body interface on which the first connection interface 500a is located. Thus, all of the devices can be simultaneously powered. Regardless of the manner in which the first connection interface 500a and the second connection interface 500b rotate, the contact interface 502 and the contact interface 506 are always aligned and in contact, given that the centers of the contact interface 502 and the contact interface 506 align with the centers of the first connection interface 500a and the second connection interface 500b. Thus, the devices can always be powered.

The first connection interface 500a further includes contact interfaces 504a-504d operatively connected to a controller (e.g., a microprocessor or another suitable processing unit) and are configured to communicate operational instructions to a respective device. The first connection interface 500a and the second connection interface 500b are configured to rotate relative to one another in the manner described with respect to the first connection interface 400a and the second connection interface 400b. The devices can be selectively activated according to the rotation of the first connection interface 500a and the second connection interface 500b (e.g., the alignment and contact of the contact interface 508 with one of the contact interfaces 504a-504d) in the manner described with respect to the contact interfaces 406a and 402a-402d.

The first connection interface 500a further includes a component 505a, 505b, 505c, or 505d for each of the devices configured to fire collected data onto a data transmission ring (e.g., a fiber optics ring 510) on the second connection interface 500b. The fiber optics ring 510 is operatively coupled to the controller and can relay the collected data to the controller 132. This enables one or more activated ones of the devices to send data to the controller 510 regardless of the relative position of the first connection interface 500a and the second connection interface 500b. This further enables multiple activated devices to send data to the fiber optics ring 510 simultaneously. For example, two or more devices are activated simultaneously (e.g., not selectively activated). Thus, the two or more devices can simultaneously fire collected data to the fiber optics ring 510 to be relayed to the controller 132.

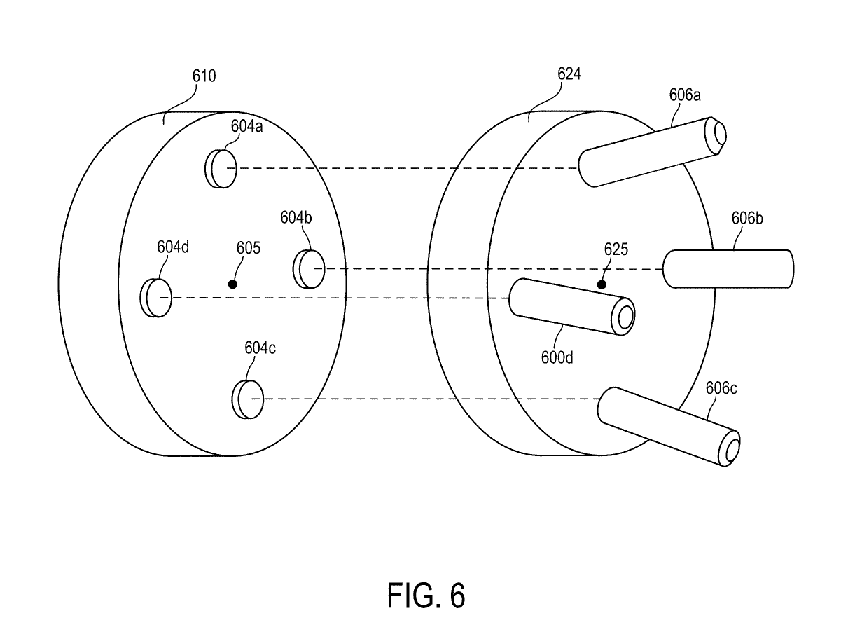

FIG. 6 illustrates tools 606a-606d being operatively coupled to devices 604a-604d according to various arrangements. Referring to FIG. 106, the tools 606a-606d are example implementations of the tools 106a-106n. The devices 604a-604d are example implementations of the devices 104a-104n. The tools 606a-606d are shown to be disposed on a platform 624, which is an example implementation of the platform 124. The devices 604a-604d are shown to be disposed on a body interface 610, which is an example implementation of the body interface 110. The platform 624 may be detachably mounted to the body interface 610 via magnets, spring-loaded pins, clamps, locks, latches, bolts, buckles, clips, hook-and-look fasteners, pegs, pins, rivets, snap fasteners, straps, and another suitable mechanical fastener. The tools 606a-606d are no longer needed when the operations of the device having data characteristics that can be modified by the tools 606a-606d are completed. The platform 624 can be automatically or manually detached from the body interface 610, to allow other devices to be activated and/or to operate.

A movement mechanism (e.g., a motor assembly) on the body interface 610 is configured to rotate the body interface 610 and/or the platform 624 about centers 605 and 625. Assembling the body interface 610 and the platform 624 includes rotatably supporting the body interface 610 and the platform 624 such that the centers 605 and 625 are aligned. As shown, the body interface 610 and the platform 624 have a same shape (e.g., a circular surface). The tools 606a-606d and the devices 604a-604d can be rotated about an axis through the centers 605 and 625. The axis can be perpendicular to the surfaces of the body interface 610 and the platform 624. In some arrangements, a center (e.g., the center 410b) of the second connection interface (e.g., the second connection interface 400b) is also on the axis, such that the body interface 610, the platform 624, and the second connection interface are configured to rotate about a same axis.

Examples of the devices 604a-604d include but are not limited to, a photo-sensor, an infrared sensor, a particle detector, a laser emitter, and the like. Examples of the tools 606a-606d include but are not limited to, a lens, a photo-projector, a filter configured to filter or pass designated light or laser frequencies, an amplifier that amplifies a signal, an attenuator that attenuates a signal, and a waveguide.

In some arrangements, the device 604a is a photo-sensor (e.g., a complementary metal-oxide-semiconductor (CMOS)), and the tool 606a is a wide-angle lens. The tool 606a is operatively coupled to the device 604a when the tools 606a-606d (and the platform 624) are in a first position relative to the devices 604a-604d (and the body interface 110), causing the tool 606a to align with the device 604a. The first data generated by the device 604a (as modified by the tool 606a) is first image data capturing a first portion of a subject. Given that the tool 606a is a wide-angle lens, the device 604a can capture a larger portion of the subject. The tool 606b is a zoom lens. The tool 606b is operatively coupled to the device 604a when the tools 606a-606d (and the platform 624) are in a second position relative to the devices 604a-604d (and the body interface 110), causing the tool 606b to align with the device 604a. The second data generated by the device 604a (as modified by the tool 606b) is second image data capturing a second portion of a subject. Given that the tool 606a is a zoom lens, the device 604a can capture a smaller portion of the subject, with improved resolution. In that regard, the tool 606a can be used in connection with the device 604a to perform a full-body imaging or 3D imaging of the subject, the second tool 606b can be used in connection with the device 604a to perform localized or target imaging for a particular area of the body of the subject.

In some arrangements, the device 604c is a laser emitter, and the tool 606c and the tool 606d are grid lenses or waveguides configured to modify laser characteristics such that a laser grid can be projected, for example onto the subject by the device 604c. The tool 606c is operatively coupled to the device 604c when the tools 606a-606d (and the platform 624) are in the first position relative to the devices 604a-604d (and the body interface 110), causing the tool 606c to align with the device 604c. The devices 604a and 604c can be simultaneously activated such that in the first position, the wide-angle images collected by the devices 604a (as modified by the tool 606a) capture the subject with the laser grid projected thereon, for example, to assist any automatic registration of the subject or another one of the devices 604a-604d. Automatic registration refers to defining a workspace of the devices 604a-604n and the tools 606a-606n by defining boundaries on the body of the subject within which the devices 604a-604n and the tools 606a-606n operate. For example, imaging devices can capture images or videos of the subject, including anatomical landmarks or features of the subject. The tool 606d is operatively coupled to the device 604c when the tools 606a-606d (and the platform 624) are in the second position relative to the devices 604a-604d (and the body interface 110), causing the tool 606d to align with the device 604c. The devices 604a and 604c can be simultaneously activated such that in the second position, the zoom images collected by the devices 604a (as modified by the tool 606b) capture the portion of the subject with the laser grid projected thereon. The laser grid can be used as reference lines to segment the body of the subject or a portion thereof to assist operations or registration of the devices 604a-604n and the tools 606a-606n, for example, by defining paths or spaces on the body, determining 3D characteristics across the body to image depths of the body, and the like.

In another example involving simultaneous operation of the devices and tools, the device 604c is a light emitter (e.g., a black light emitter, a non-visible light emitter configured to facilitate identification or portions or feature of the body, including veins, such that other devices such as a syringe can be operated based on images captured under the light emitter), and the tool 606c is a black light lens configured to modify (e.g., focus) the black light emitted by the device 604c. The tool 606c is operatively coupled to the device 604c when the tools 606a-606d (and the platform 624) are in the first position relative to the devices 604a-604d (and the body interface 110), causing the tool 606c to align with the device 604c. The devices 604a and 604c can be simultaneously activated such that in the first position, the wide-angle images collected by the devices 604a (as modified by the tool 606a) capture the subject under black light.

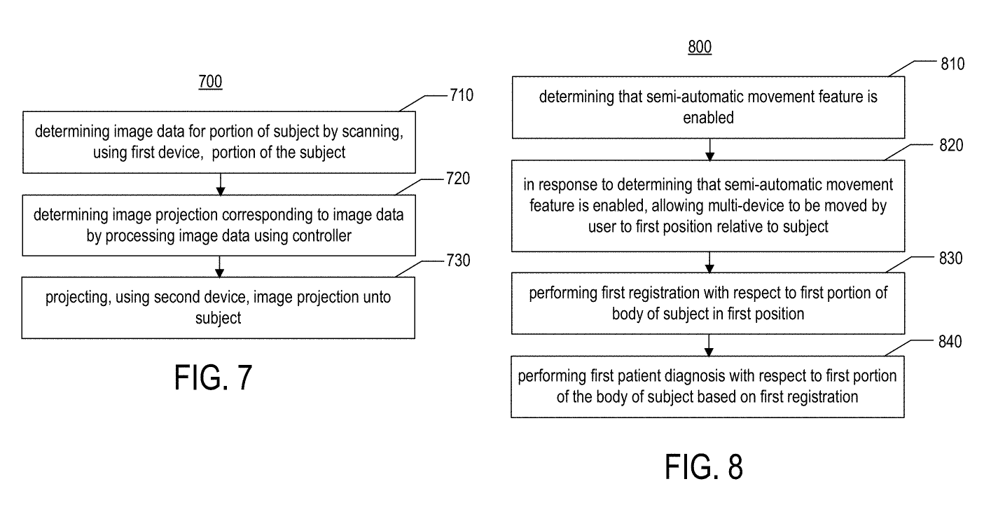

FIG. 7 is a flow diagram illustrating a method 700 for using the multi-device system 100 according to various arrangements. Referring to FIGS. 1-7, the device 104a (e.g., an ultrasound image device or another suitable image device) determines imaging data for a portion of a subject by scanning the portion of the subject, at 710. For example, the device 104a collects ultrasound data of a heart valve of the subject. At 720, the controller 132 determines an image projection corresponding to the imaging data by processing the imaging data. Processing includes at least generating an image of the heart valve of the subject based on the ultrasound data in some examples. At 730, the device 104b (e.g., a projector) is configured to project the image projection unto the subject. The image projection allows a healthcare provider to visualize the portion of the subject's body that has been imaged, for example, using ultrasound. Images are updated in real-time such that, for example, the healthcare provider can visualize real-time physical changes (e.g., a beating heart, changes due to surgery, and the like). For example, the healthcare provider can perceive images or videos of bone or vasculature in real-time, or other organ/tissue. The image projection is projected unto the same portion (e.g., unto a skin of the subject that is proximate to the heart valve) of the subject or unto a different portion of the subject, to facilitate healthcare. In some arrangements, the method 700 further includes performing registration with respect to the portion (e.g., the heart) of the subject using the device 104c. Registration refers to determining a position corresponding to the portion of interest of the subject by a camera or photo-sensor such that the device 104a can be configured to generate the imaging data at the position.

FIG. 8 is a flow diagram illustrating a method 800 for using the multi-device system 100 according to various arrangements. Referring to FIGS. 1-8, the controller 132 determines that a semi-automatic movement feature is enabled, at 810. For example, the controller 132 can determine that the semi-automatic movement feature is enabled responsive to determining that the semi-automatic switch 148 is in the first state.

At 820, in response to determining that the semi-automatic movement feature is enabled, the multi-device 102 is allowed to be moved by a user (e.g., a healthcare provider) to a first position relative to the subject. For example, responsive to determining that the semi-automatic movement feature is enabled, the controller 132 is configured to loosen one or more joints (e.g., the joints 306a and 306b) on the body 130 (302), such that the user can move the multi-device 102 (200) by moving the manual interface 150. The user can bring the multi-device 102 closer to the first portion by moving the manual interface 150. Such arrangements conserve time needed to execute a full-body registration to identify the first portion and to automatically move the multi-device 102 close to the first portion. For example, in a procedure involving echocardiogram, the user can enable the semi-automatic movement feature and push the multi-device 102 (via the manual interface 150) toward a chest of the subject.

At 830, the multi-device 102 (e.g., the device 104a, which may be a camera or a photo-sensor) performs a first registration with respect to a first portion of a body of the subject in the first position. The controller 132 automatically adjusts the first position after the multi-device 102 has been moved closer to the first portion by the user, to position the multi-device 102 in the optimal data acquisition position for subject imaging or diagnosis. In other words, the registration process more finely defines or identifies the first portion on which the subject diagnosis is to be performed. The first registration process refines the first position.

At 840, the multi-device 102 (e.g., the device 104b) performs first subject diagnosis with respect to the first portion of the body of the subject based on the first registration. The first subject diagnosis includes any suitable procedure described herein.

In some arrangements, the method 800 further includes allowing the multi-device 102 to be moved by the user to a second position relative to the subject, after the first subject imaging or diagnosis is performed. From the first position, the user can bring the multi-device 102 closer to a second portion by moving the manual interface 150. The multi-device 102 (e.g., the device 104a) performs a second registration with respect to the second portion of the body of the subject in the second position, in the manner described similar to the first registration process. The second subject imaging or diagnosis with respect to the second portion of the body of the subject can be performed based on the second registration, in the manner described similar to the first subject diagnosis.

In some arrangements, the first portion is larger in size than the second portion. In some examples, the processes (e.g., 830-840) with respect to the first portion are full body registration and diagnosis processes. The full body registration and diagnosis processes may identify potential concerns in, for example, the second portion, which may be a part of the first portion. In that regard, the second registration is performed based on a smaller workspace as compared to that of the first registration. In some example, the first registration identifies an area of the body of the subject to be focused on, and the second registration corresponds to the area identified by the first registration.

In some arrangements, the first portion and the second portion correspond to different body parts of the subject. This allows the multi-device system 100 to perform automatic registration and imaging or diagnosis processes for different body parts (e.g., organs).

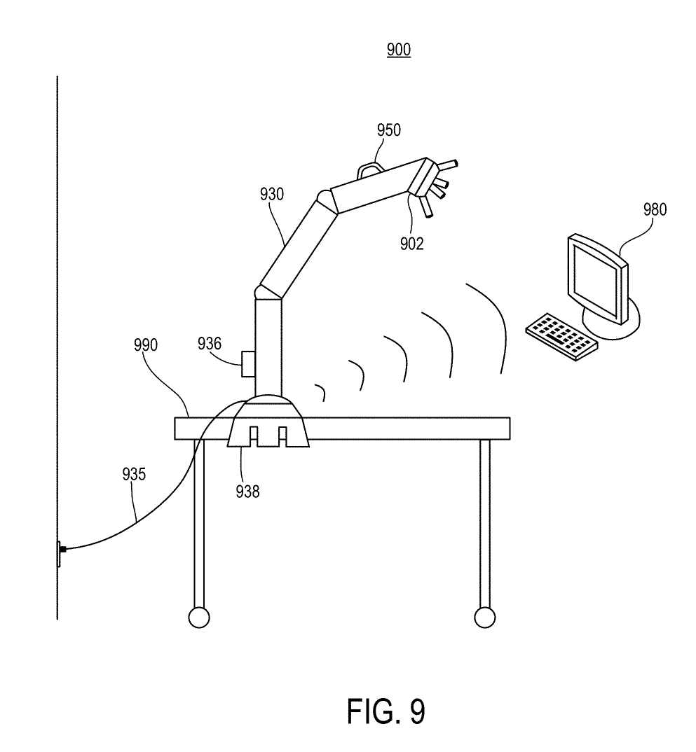

FIG. 9 illustrates a multi-device system 900 according to various arrangements. The multi-device system 900 is an example implementation of the multi-device system 100. Referring to FIGS. 1-9, the multi-device system 900 includes a multi-device 902, a body 930, a manual interface 950, and a mounting mechanism 938, each of which corresponds to a respective one of the multi-device 102, the body 130, the manual interface 150, the mounting mechanism 138. In particular, the multi-device 902 is arranged on an end of the body 930, which is a robotic manipulator having arms and joints. The manual interface 950 is a handle arranged on an arm segment of the body 930 closest to the multi-device 902. The mounting mechanism 938 is a clamp configured to mount the multi-device system 900 to a movable desk. The multi-device system 900 includes a power supply 934 or a power cord 935. The power supply 934 and the power cord 935 are example implementations of the power supply 134. The power supply 934 is a battery arranged on the body 930, such that an external power source is not needed. The power supply 934 is rechargeable and/or replaceable. The power cord 935 can receive power from an external power source (e.g., an electrical outlet). The body 130 includes a network device (not shown) corresponding to the network device 136 and is configured to receive operational instructions from a computing platform 980 and/or send data to the computing platform 980.

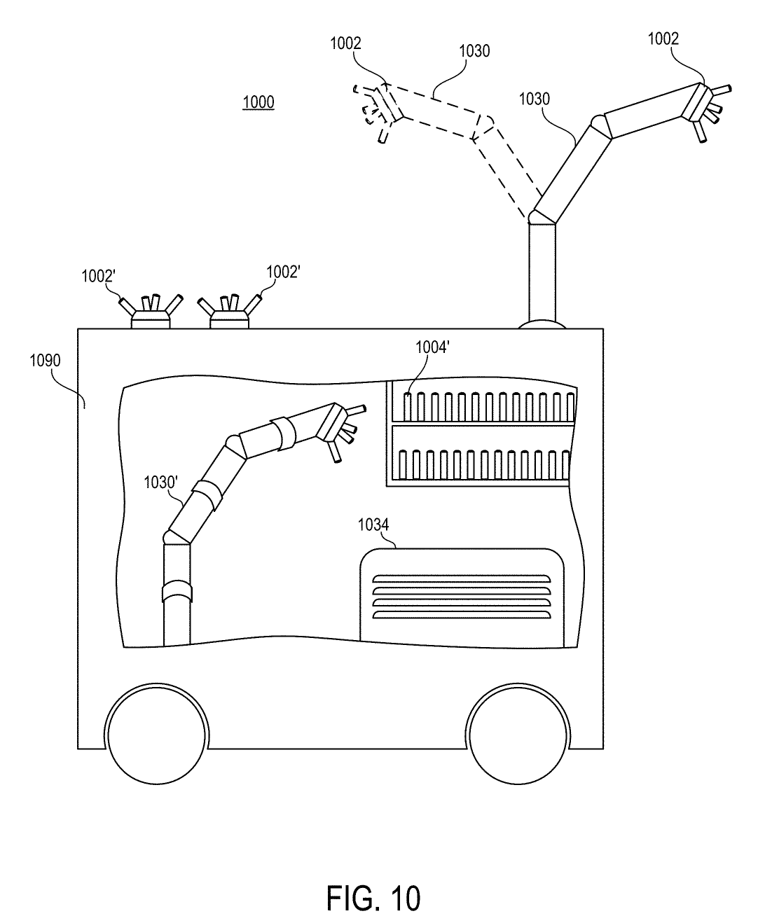

FIG. 10 illustrates a multi-device system 1000 according to various arrangements. The multi-device system 1000 is an example implementation of the multi-device system 100. Referring to FIGS. 1-10, the multi-device system 1000 includes a multi-device 1002 and a body 1030, each of which corresponds to a respective one of the multi-device 102 and the body 130. In particular, the multi-device 1002 is arranged on an end of the body 1030, which is a robotic manipulator having arms and joints. The body 1030 is attached to a movable cart 1090 having an internal storage area. The internal storage area is configured to store at least one replacement body 1030′, a power supply 1034 (corresponding to the power supply 134), and replacement devices 1004′. Each of the at least one replacement body 1030′ can have different arm lengths, number of joints, functions, and the like, and can be implemented in accordance with the desired use. In one example, the replacement body 1030′ may have more joints for finer movements within the workspace. The replacement multi-devices 1002′ can have different devices or a combination of devices as compared to the devices of the multi-device 1002. For example, one of the replacement multi-devices 1002′ can have diagnostic devices arranged thereon, and another one of the replacement multi-devices 1002′ can have therapeutic devices arranged thereon, where the replacement multi-devices 1002′ can be swapped based on implementation and desired use.

The cart 1090 has an upper surface configured to hold replacement multi-devices 1002′. A user can replace the multi-device 1002 with one of the replacement multi-devices 1002′ manually, replace the devices on the multi-device 1002 with the replacement devices 1004′, and replace the body 1030 with the replacement body 1030′ manually. In other examples, the body 1030 can move the multi-device 1002 over the upper surface of the cart 1090, automatically detach or otherwise release the multi-device 1002, and attach the replacement multi-device 1002. Similarly, the body 1030 can move the multi-device 1002 into the internal storage area of the cart 1090, automatically detach or otherwise release one or more devices on the multi-device 1002, and attach to the replacement devices 1004′. In other examples, the replacement multi-devices 1002′ can be stored in the internal storage area and replaced in a manner similar to described with respect to the replacement devices 1004′. The replacement devices 1004′ can be disposed on the upper surface and replaced in a manner similar to described with respect to the replacement multi-devices 1002′. The tools 106a-106n can be stored and replaced in a similar manner.

While the arrangements herein show one multi-device operatively coupled to a body, multiple multi-devices can be arranged on a same body. For example, one body may include multiple branch arms, an end of each branch arm is operatively coupled to a multi-device in the manner described. In addition, multiple bodies each having at least one multi-device provided thereon can be similarly implemented. The multi-devices can be simultaneously activated to perform a joint task. For example, a device (e.g., an ultrasound device) on each of multiple multi-devices can be pointed to a kidney stone or blood clot on the subject to focus energy to jointly break down the kidney stone or blood clot. In another example, a first device (e.g., a lighting device) of a first multi-device illuminates at least a portion of the subject while a second device (e.g., a laser emitter modified by a waveguide tool) of a second multi-device projects a laser grid onto the at least a portion of the subject, while a third device (e.g., a photo-sensor) of a third multi-device generates images of the at least one portion of the subject.

The above used terms, including “held fast,” “mount,” “attached,” “coupled,” “affixed,” “connected,” “secured,” and the like are used interchangeably. In addition, while certain arrangements have been described to include a first element as being “coupled” (or “attached,” “connected,” “fastened,” etc.) to a second element, the first element may be directly coupled to the second element or may be indirectly coupled to the second element via a third element.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but are to be accorded the full scope consistent with the language, wherein reference to an element in the singular is not intended to mean “one and only one” unless specifically so stated, but rather “one or more.” Unless specifically stated otherwise, the term “some” refers to one or more. All structural and functional equivalents to the elements of the various aspects described throughout the previous description that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed as a means plus function unless the element is expressly recited using the phrase “means for.”

It is understood that the specific order or hierarchy of steps in the processes disclosed is an example of illustrative approaches. Based upon design preferences, it is understood that the specific order or hierarchy of steps in the processes may be rearranged while remaining within the scope of the previous description. The accompanying method claims present elements of the various steps in a sample order, and are not meant to be limited to the specific order or hierarchy presented.

The previous description of the disclosed implementations is provided to enable any person skilled in the art to make or use the disclosed subject matter. Various modifications to these implementations will be readily apparent to those skilled in the art, and the generic principles defined herein may be applied to other implementations without departing from the spirit or scope of the previous description. Thus, the previous description is not intended to be limited to the implementations shown herein but is to be accorded the widest scope consistent with the principles and novel features disclosed herein.

What is claimed is:

1. A multi-device system for providing healthcare to a subject, comprising:
a multi-device comprising:
a body interface;
a plurality of devices fixed to the body interface, each of the plurality of devices configured to collect data of or provide therapy to the subject; and
a body operatively coupled to the multi-device at the body interface, wherein the body is configured to position the multi-device with respect to the subject, and the body interface is configured to move relative to the body to selectively activate at least one of the plurality of devices.

2. The multi-device system of claim 1, wherein each of the plurality of devices is configured to collect data of the subject and comprises at least one of a camera, an ultrasound range device, an ultrasound imaging device, a thermal imaging device, a near-infrared imaging device, a near-infrared spectroscopy device, an optical camera device, a lighting device, a spectrometer device, a microphone device, an electroencephalography device, and a metal detector.

3. The multi-device system of claim 1, wherein each of the plurality of devices is configured to provide therapy to the subject and comprises at least one of a laser, a syringe, an ablation device, a light therapy device, an electric shock therapy device, a stitching device, a scalpel, a catheter, an insulin pump, a bandaging device, and a respirator.

4. The multi-device system of claim 1, further comprising:
a power supply configured to provide power to the plurality of devices; and
a controller configured to transmit operational instructions to the plurality of devices and receive the data collected by the plurality of devices,
wherein the at least one of the plurality of devices is activated when the at least one of the plurality of devices is receiving the power and the operational instructions.

5. The multi-device system of claim 4, wherein
the body interface comprises a first connection interface operatively coupled to each of the plurality of devices;
the body comprises a second connection interface operatively coupled to the power supply and the controller;
the second connection interface is configured to provide the power and the operational instructions to the first connection interface for the activated at least one of the plurality of devices by operatively coupling to the first connection interface when the body interface is moved relative to the body to a predetermined position; and
the first connection interface is configured to provide the data collected by the at least one of the plurality of devices to the second connection interface by operatively coupling to the second connection interface.

6. The multi-device system of claim 5, wherein
the first connection interface for the at least one of the plurality of devices comprises a first power interface and a first data communication interface separate from the first power interface;
the second connection interface comprises a second power interface and a second data communication interface separate from the second power interface;
the second power interface is configured to operatively couple to the first power interface when the body interface is moved relative to the body to the predetermined position to provide the power to the first power interface; and
the second data communication interface is configured to operatively couple to the first data communication interface when the body interface is moved relative to the body to the predetermined position to provide the operational instructions to the first data communication interface and to receive the data collected by the activated at least one of the plurality of devices from the first data communication interface.

7. The multi-device system of claim 5, wherein the second connection interface is configured to provide the power and the operational instructions to the first connection interface by providing to the first connection interface modulated power signals comprising the power from the power supply and the operational instructions from the controller.

8. The multi-device system of claim 5, wherein the body interface comprises a locking device configured to structurally maintain the predetermine position.

9. The multi-device system of claim 4, wherein
the body interface comprises a first connection interface operatively coupled to each of the plurality of devices;
the body comprises a second connection interface operatively coupled to the power supply and the controller;
the second connection interface comprises at least one data transmission ring configured to receive the data collected by the at least one of the plurality of devices.

10. The multi-device system of claim 1, wherein the body interface being configured to move relative to the body to selectively activate at least one of the plurality of devices by:
responsive to the body interface being moved relative to the body to a first predetermined position, a first device of the plurality of devices is activated while a second device of the plurality of devices is inactivated; and
responsive to the body interface being moved relative to the body to a second predetermined position, the second device is activated while the first device is inactivated.

11. The multi-device system of claim 1, wherein the body interface is configured to rotate relative to the body to selectively activate the at least one of the plurality of devices.

12. The multi-device system of claim 1, wherein the at least one of the plurality of devices comprises a force controller configured to determine a force exerted by the at least one of the plurality of devices against the subject.

13. The multi-device system of claim 1, wherein
the multi-device further comprises a plurality of tools;
the plurality of tools is configured to move relative to the plurality of devices;
a first tool of the plurality of tools is configured to operatively couple to one of the at least one of the plurality of devices when the plurality of tools is moved to a first relative position relative to the plurality of devices;
the first tool being operatively coupled to the one of the at least one of the plurality of devices enables the one of the at least one of the plurality of devices to output first data;
a second tool of the plurality of tools is configured to operatively couple to the one of the at least one of the plurality of devices when the plurality of tools is moved to a second relative position relative to the plurality of devices; and
the second tool being operatively coupled to the one of the at least one of the plurality of devices enables the one of the at least one of the plurality of devices to output second data different from first data.

14. The multi-device system of claim 13, wherein
the one of the plurality of devices is one of a photo-sensor, an infrared sensor, a particle detector, and a laser emitter; and
each of the plurality of tools is at least one of a lens, a photo-projector, and a waveguide.

15. The multi-device system of claim 13, wherein
the one of the plurality of devices is a photo-sensor;
the first tool is a wide-angle lens;
the first data comprise first image data capturing a first portion of the subject;
the second tool is a zoom lens;
the second data comprise second image data capturing a second portion of the subject; and
the first portion is larger than the second portion.

16. The multi-device system of claim 15, wherein
another one of the plurality of devices is a laser emitter;
the plurality of tools comprises a grid lens configured to be operatively coupled to the laser emitter in the first relative position; and
the laser emitter is configured to project a grid onto the subject while the first data is being collected by the photo-sensor through the wide-angle lens or while the second data is being collected by the photo-sensor through the zoom lens.

17. The multi-device system of claim 1, wherein
the body comprises a robotic manipulator having at least two arm segments and a joint; and
the robotic manipulator is configured to position the multi-device with respect to the subject.

18. The multi-device system of claim 1, wherein the body further comprises at least one of:
a mounting mechanism configured to detachably mount the multi-device system to an object;
a battery; and
a network device.

19. The multi-device system of claim 1, wherein the body further comprises a manual interface configured to allow the multi-device to be moved relative to the subject by a user of the multi-device system.

20. The multi-device system of claim 19, wherein
the body further comprises a semi-automatic switch;
the semi-automatic switch being in a first state corresponds to allowing the multi-device to be moved relative to the subject by the user via the manual interface; and
the semi-automatic switch being in a second state corresponds to disallowing the multi-device to be moved relative to the subject by the user.

21. A method for providing healthcare to a subject using a multi-device system, the method comprising:
providing a multi-device comprising:
a body interface;
a plurality of devices fixed to the body interface, wherein each of the plurality of devices is configured to provide healthcare to the subject by collecting data; and
providing a body operatively coupled to the multi-device at the body interface, wherein the body is configured to position the multi-device with respect to the subject, and the body interface is configured to move relative to the body to selectively activate at least one of the plurality of devices.

22. The method of claim 21, further comprising:
providing a plurality of tools, wherein
the plurality of tools is configured to move relative to the plurality of devices;
a first tool of the plurality of tools is configured to operatively couple to a first device of the at least one of the plurality of devices when the plurality of tools is moved to a first relative position relative to the plurality of devices;
the first tool being operatively coupled to the first device enables the first device to output first data;
a second tool of the plurality of tools is configured to operatively couple to the first device when the plurality of tools is moved to a second relative position relative to the plurality of devices; and
the second tool being operatively coupled to the first device enables the first device to output second data different from first data.

23. The method of claim 21, wherein the first device and a second device of the at least one of the plurality of devices are configured to be activated simultaneously.

24. The method of claim 23, wherein a third tool of the plurality of tools is configured to operatively couple to the second device when the plurality of tools is moved to the first relative position relative to the plurality of devices.

25. The method of claim 24, wherein
the first device is a photo-sensor;
the first tool is a wide-angle lens;
the second device is a laser emitter; and
the second tool is a grid lens.

26. A method for providing healthcare to a subject using multi-device system having a multi-device and a body configured to move the multi-device relative to the subject, the method comprising:
determining that a semi-automatic movement feature is enabled;
in response to determining that the semi-automatic movement feature is enabled, allowing the multi-device to be moved by a user to a first position relative to the subject;
performing first registration with respect to a first portion of a body of the subject in the first position; and
performing first subject imaging with respect to the first portion of the body of the subject based on the first registration.

27. The method of claim 26, wherein allowing the multi-device to be moved relative to the subject by the user comprises:
providing a manual interface on the body or the multi-device, wherein the manual interface is configured to interact with the user to allow the user to move the multi-device; and
enabling the user to move the multi-device by loosening one or more joints on the body.

28. The method of claim 26, further comprising:
allowing the multi-device to be moved by the user to a second position relative to the subject;
performing second registration with respect to a second portion of a body of the subject in the second position; and
performing second subject imaging with respect to the second portion of the body of the subject based on the second registration.

29. A method for providing healthcare to a subject using a multi-device system having a controller, a multi-device, and a body configured to move the multi-device relative to the subject, the multi-device having a first device and a second device, the method comprising:
determining imaging data for a portion of the subject by scanning, using the first device, the portion of the subject;
determining image projection corresponding to the imaging data by processing the imaging data using the controller; and
projecting, using the second device, the image projection unto the subject.

30. The method of claim 31, wherein the image projection is projected using the second device unto the portion of the subject or a different portion of the subject.
